(19) United States
(12) Patent Application Publication
(10) Pub. No.: US 2019/0150445 A1
(43) Pub. Date: May 23, 2019

(54) SUBSTITUTED HALOGEN(THIO)ACYL COMPOUNDS

(71) Applicant: Bayer CropScience Aktiengesellschaft, Monheim am Rhein (DE)

(72) Inventors: Peter JESCHKE, Bergisch Gladbach (DE); Elke HELLWEGE, Langenfeld (DE); Reiner FISCHER, Monheim (DE); Peter LOESEL, Leverkusen (DE); Sascha EILMUS, Leichlingen (DE); Kerstin ILG, Koeln (DE); Daniela PORTZ, Vettweiss (DE); Ulrich GOERGENS, Ratingen (DE); Andreas TURBERG, Haan (DE)

(21) Appl. No.: 16/320,733

(22) PCT Filed: Jul. 21, 2017
(86) PCT No.: PCT/EP2017/068454

§ 371 Date: Jan. 25, 2019

(30) Foreign Application Priority Data
Jul. 29, 2016 (EP) 16181828.1

(51) Int. Cl. A01N 43/78; C07D 417/06; C07D 401/06; A01N 43/50

(52) U.S. Cl. CPC A01N 43/78; A01N 43/50; C07D 401/06; C07D 417/06

(57) ABSTRACT

The present invention relates to novel substituted halogen(thio)acyl compounds according to formula(I), to processes for their preparation and to their use for controlling animal pests, especially arthropods, in particular insects.

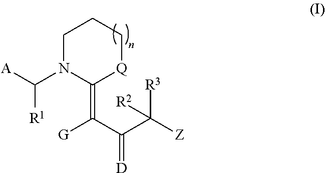

The present invention relates to novel substituted halogen(thio)acyl compounds, to processes for their preparation and to their use for controlling animal pests, especially arthropods, in particular insects.

Certain substituted halogenacyl compounds are already known as insecticidally and ectoparacidally active compounds (cf. EP 0 268 915 A1, 1995). These compounds exhibit heterocyclic radicals that are only non- or mono-substituted. Surprisingly, the introduction of heterocyclic radicals (A group) with at least two substitutents other than hydrogen significantly increases the activity of such compounds as pesticides.

This invention now provides novel compounds of the formula (I),

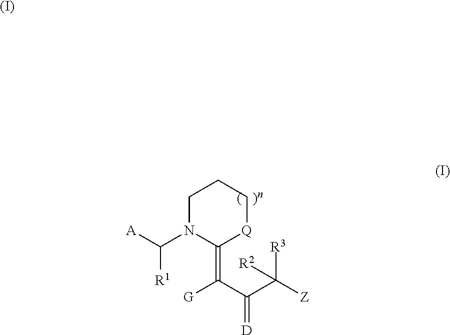

in which
A represents a pyridinyl, pyrimidinyl, pyrazolyl, thiophenyl, oxazolyl, isoxazolyl, 1,2,4-oxa-diazolyl, thiazolyl, isothiazolyl, 1,2,4-triazolyl or 1,2,5-thiadiazolyl radical which is substituted by at least one fluorine, chlorine, bromine, cyano, nitro, C₁-C₄-alkyl (which is optionally substituted by fluorine and/or chlorine), C₁-C₃-alkylthio (which is optionally substituted by fluorine and/or chlorine), or C₁-C₃-alkylsulphonyl (which is optionally substituted by fluorine and/or chlorine),
n represents 0 or 1
D represents sulphur,
G represents hydrogen,
Q represents sulphur, oxygen or NH,
R¹ represents hydrogen or alkyl,
R² represents halogen,
R³ represents halogen,
Z represents hydrogen, halogen or halogenalkyl,

or
A represents a radical

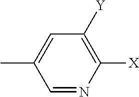

in which
X represents halogen, alkyl or haloalkyl,
Y represents halogen, alkyl, haloalkyl, haloalkoxy, azido or cyano,
n represents 0 or 1
D represents oxygen or sulphur,
G represents hydrogen or halogen,
Q represents sulphur, oxygen or NH,
R¹ represents hydrogen or alkyl,
R² represents halogen,
R³ represents halogen,
Z represents hydrogen, halogen or halogenalkyl.

Finally, it has been found that the novel compounds of the formula (I) have pronounced biological properties and are suitable especially for controlling animal pests, in particular insects, arachnids and nematodes encountered in agriculture, in forests, in the protection of stored products and in the protection of materials, and also in the hygiene sector.

Depending inter alia on the nature of the substituents, the compounds of the formula (I) may be present as geometrical and/or as optically active isomers or corresponding isomer mixtures of varying composition. The invention relates both to the pure isomers and the isomer mixtures.

The formula (I) provides a general definition of the compounds according to the invention.

Preferred substituents or ranges of the radicals given in the formulae mentioned above and below are illustrated below.

In a two preferred groups of compounds of formula (I) either
A preferably represents pyrimidin-5-yl which is optionally substituted in the 2-position by halogen or C₁-C₄-alkyl, represents 1H-pyrazol-4-yl which is optionally substituted in the 1-position by C₁-C₄-alkyl and in the 3-position by halogen, represents 1H-pyrazol-5-yl which is optionally substituted in the 2-position by halogen or C₁-C₄-alkyl, represents isoxazol-5-yl which is optionally substituted in the 3-position by halogen or C₁-C₄-alkyl, represents 1,2,4-oxadiazol-5-yl which is optionally substituted in the 3-position by halogen or C₁-C₄-alkyl, represents 1-methyl-1,2,4-triazol-3-yl or represents 1,2,5-thiadiazol-3-yl, and
n preferably represents 0 or 1, and
D preferably represents sulfur, and
G preferably represents hydrogen, and
Q preferably represents sulphur or NH, and
R¹ preferably represents hydrogen, and
R² preferably represents halogen, and
R³ preferably represents halogen, and
Z preferably represents halogen or halogenalkyl;
   or
A preferably represents one of the radicals 5,6-difluoropyrid-3-yl, 5-chloro-6-fluoropyrid-3-yl, 5-bromo-6-fluoropyrid-3-yl, 5-iodo-6-fluoropyrid-3-yl, 5-fluoro-6-chloropyrid-3-yl, 5,6-dichloropyrid-3-yl, 5-bromo-6-chloropyrid-3-yl, 5-iodo-6-chloropyrid-3-yl, 5-fluoro-6-bromopyrid-3-yl, 5-chloro-6-bromopyrid-3-yl, 5,6-dibromopyrid-3-yl, 5-iodo-6-bromopyrid-3-yl, 5-fluoro-6-iodopyrid-3-yl, 5-chloro-6-iodopyrid-3-yl, 5-bromo-6-iodopyrid-3-yl, 5,6-diiodopyrid-3-yl, 5-methyl-6-fluoropyrid-3-yl, 5-methyl-6-chloropyrid-3-yl, 5-methyl-6-bromopyrid-3-yl, 5-methyl-6-iodopyrid-3-yl, 5-difluoromethyl-6-fluoropyrid-3-yl, 5-difluoromethyl-6-chloropyrid-3-yl, 5-difluoromethyl-6-bromopyrid-3-yl, 5-difluoromethyl-6-iodopyrid-3-yl, and
n preferably represents 0 or 1, and
D preferably represents oxygen, and
G preferably represents hydrogen, and
Q preferably represents sulphur or NH, and
R¹ preferably represents hydrogen, and
R² preferably represents halogen, and
R³ preferably represents halogen, and
Z preferably represents halogen or halogenalkyl.

Particularly preferred groups of compounds according to formula (I):
A particularly preferably represents 5-fluoro-6-chloropyrid-3-yl, 5,6-dichloropyrid-3-yl, 5-bromo-6-chloropyrid-3-yl, 5-fluoro-6-bromopyrid-3-yl, 5-chloro-6-bromopyrid-3-yl, 5,6-dibromopyrid-3-yl, 5-methyl-6-chloropyrid-3-yl or 5-methyl-6-bromopyrid-3-yl.
n particularly preferably represents 0
D particularly preferable represents oxygen,
G particularly preferable represents hydrogen,
Q particularly preferable represents sulphur or NH,
R¹ particularly preferable represents hydrogen,
R² particularly preferable represents halogen,
R³ particularly preferable represents halogen,
Z particularly preferable represents halogen.

Very particularly preferred groups of compounds according to formula (I):
A very particularly preferably represents 5-fluoro-6-chloropyrid-3-yl, 5,6-difluoro-pyrid-3-yl or 5-fluoro-6-bromopyrid-3-yl.
n very particularly preferably represents 0
D very particularly preferable represents oxygen,
G very particularly preferable represents hydrogen,
Q very particularly preferable represents NH,
R¹ very particularly preferable represents hydrogen,
R^{Z} very particularly preferable represents halogen,
R³ very particularly preferable represents halogen,
Z very particularly preferable represents halogen.

In a further special group of compounds of the formula (I), A represents 5-fluoro-6-chloropyrid-3-yl,

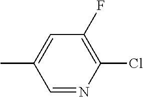

In a further special group of compounds of the formula (I), A represents 5,6-difluoro-pyrid-3-yl,

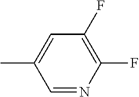

In a further special group of compounds of the formula (I), A represents 5,6-dichloropyrid-3-yl

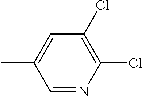

In a further special group of compounds of the formula (I), A represents 5-fluoro-6-bromopyrid-3-yl

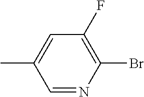

In a further special group of compounds of the formula (I), A represents 5-fluoro-6-iodopyrid-3-yl

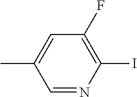

In a further special group of compounds of the formula (I), A represents 5-chloro-6-iodopyrid-3-yl

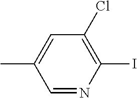

A further group of preferred compounds of the formula (I) is defined in which
A represents a radical

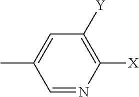

in which
X represents halogen or halo-C₁-C₄-alkyl,
Y represents halogen, C₁-C₄-alkyl, halo-C₁-C₄-alkyl, halo-C₁-C₄-alkoxy, azido or cyano,
n represents 0 and 1,
Q represents sulphur and NH,
D represents oxygen,
G represents hydrogen,
R¹ represents hydrogen,
R² represents halogen,
R³ represents halogen,
Z represents halogen or halogenalkyl.

Additional groups of preferred, particularly preferred and very particularly preferred compounds of formula (I) are defined in which
A preferably represents one of the radicals 5,6-difluoropyrid-3-yl, 5-chloro-6-fluoropyrid-3-yl, 5-bromo-6-fluoropyrid-3-yl, 5-iodo-6-fluoropyrid-3-yl, 5-fluoro-6-chloropyrid-3-yl, 5,6-dichloropyrid-3-yl, 5-bromo-6-chloropyrid-3-yl, 5-iodo-6-chloropyrid-3-yl, 5-fluoro-6-bromopyrid-3-yl, 5-chloro-6-bromopyrid-3-yl, 5,6-dibromopyrid-3-yl, 5-fluoro-6-iodopyrid-3-yl, 5-chloro-6-iodopyrid-3-yl, 5-bromo-6-iodopyrid-3-yl, 5-methyl-6-fluoropyrid-3-yl, 5-methyl-6-chloropyrid-3-yl, 5-methyl-6-bromopyrid-3-yl, 5-methyl-6-iodopyrid-3-yl, 5-difluoromethyl-6-fluoropyrid-3-yl, 5-difluoromethyl-6-chloropyrid-3-yl, 5-difluoromethyl-6-bromopyrid-3-yl, 5-difluoromethyl-6-iodopyrid-3-yl.
n preferably represents 0 and 1,
Q preferably represents sulphur and NH,
R preferably represents hydrogen.
A particularly preferably represents 2-chloropyrimidin-5-yl, 5-fluoro-6-chloropyrid-3-yl, 5,6-dichloropyrid-3-yl, 5-bromo-6-chloropyrid-3-yl, 5-fluoro-6-bromopyrid-3-yl, 5-chloro-6-bromopyrid-3-yl, 5,6-dibromopyrid-3-yl, 5-methyl-6-chloropyrid-3-yl, 5-chloro-6-iodopyrid-3-yl or 5-difluoromethyl-6-chloropyrid-3-yl.
R¹ particularly preferably represents hydrogen.
n particularly preferably represents 0 and 1,
Q particularly preferably represents sulphur and NH.
A very particularly preferably represents 5-fluoro-6-chloropyrid-3-yl or 5-fluoro-6-bromopyrid-3-yl.
n very particularly preferably represents 0,
Q very particularly preferably represents sulphur,
R very particularly preferably represents hydrogen.
A very particularly preferably represents 5-fluoro-6-chloropyrid-3-yl or 5-fluoro-6-bromopyrid-3-yl.
n very particularly preferably represents 0,
Q very particularly preferably represents NH,
R very particularly preferably represents hydrogen.
A very particularly preferably represents 5-fluoro-6-chloropyrid-3-yl or 5-fluoro-6-bromopyrid-3-yl.
n very particularly preferably represents 1,
Q very particularly preferably represents sulphur,
R very particularly preferably represents hydrogen.
A further very particularly preferably represents 5-fluoro-6-chloropyrid-3-yl or 5-fluoro-6-bromopyrid-3-yl.
n very particularly preferably represents 1,
Q very particularly preferably represents NH,
R very particularly preferably represents hydrogen.

In a further special group of compounds of the formula (I), R represents hydrogen, and A represents 5-bromo-6-chloropyrid-3-yl

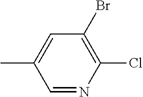

In a further special group of compounds of the formula (I), R represents hydrogen, and A represents 5-chloro-6-bromopyrid-3-yl

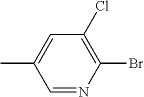

In a further special group of compounds of the formula (I), R represents hydrogen, and A represents 5-fluoro-6-chloropyrid-3-yl

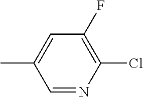

In a further special group of compounds of the formula (I), A represents 5,6-difluoro-pyrid-3-yl,

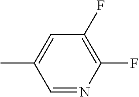

In a further special group of compounds of the formula (I), R represents hydrogen, A represents 5,6-dichloropyrid-3-yl

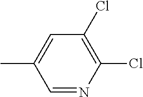

In a further special group of compounds of the formula (I), R represents hydrogen, and A represents 5-fluoro-6-bromopyrid-3-yl

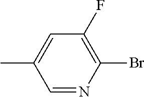

In a further special group of compounds of the formula (I), R represents hydrogen, and A represents 5-methyl-6-chloropyrid-3-yl

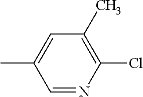

In a further special group of compounds of the formula (I), R represents hydrogen, and A represents 5-chloro-6-iodopyrid-3-yl

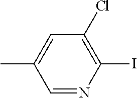

The general or preferred radical definitions given above apply both to the end products and, correspondingly, to precursors and intermediates. These radical definitions can be combined with one another as desired, i.e. including combinations between the respective preferred ranges.

Preference according to the invention is given to the compounds of the formula (I) which contain a combination of the meanings listed above as being preferred.

Particular preference according to the invention is given to the compounds of the formula (I) which contain a combination of the meanings listed above as being particularly preferred.

Very particular preference according to the invention is given to the compounds of the formula (I) which contain a combination of the meanings listed above as being very particularly preferred.

If appropriate, the compounds of the formula (I) can be present in different polymorphic forms or as a mixture of different polymorphic forms. Both the pure polymorphs and the polymorph mixtures are provided by the invention and can be used according to the invention.

The above-specified individual general, preferred, more preferred and most preferred definitions of A, G, D, Q, Z, n, R¹, R² and R³ can be combined with one another as desired in accordance with the invention. Prefered compounds according to the invention are also those of the formulae (I-A-a), (I-A-b), (I-B-a), (I-B-b), (I-D-a), (I-D-b), (I-D-c) and (I-D-d).

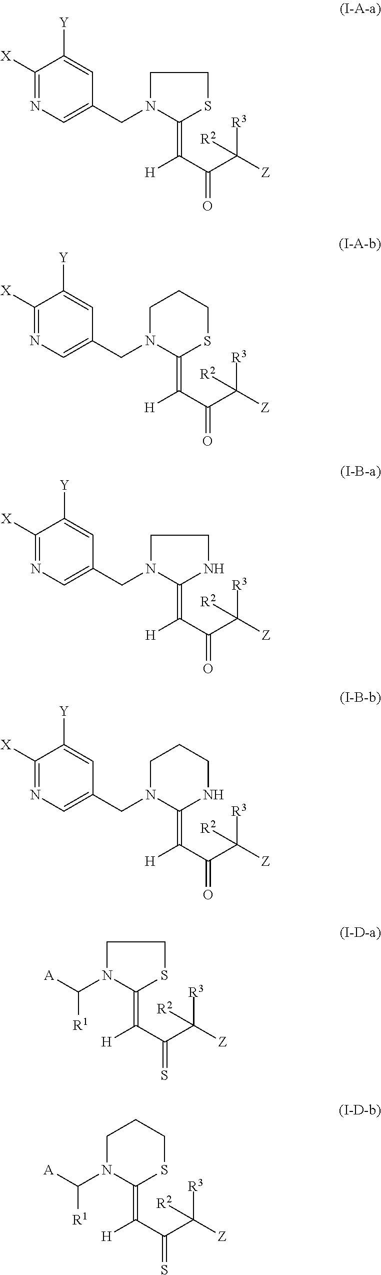

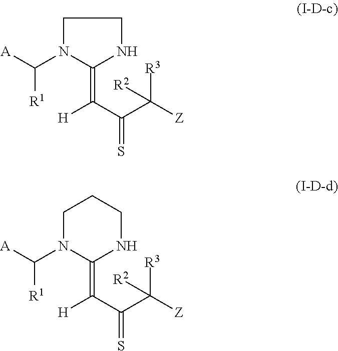

in which

A, D, Q, X, Y, Z, n, R¹, R² and R³ represent the above-mentioned general, preferred, particularly preferred and very particularly preferred definitions.

### PROCEDURES AND METHODS

One or more of the following methods A-D and variations as described in Schemes 1-4 can be used to prepare the compounds of formula (I). The definitions of A, G, D, Q, Z, n, R¹, R² and R³ in the compounds of formulae (I) below are as defined above in the Summary of the Invention unless otherwise noted. Formulae (I-A)-(I-D) are various subsets of formula (I), and all substituents for formulae (I-A)-(I-D) are as defined above for formula (I) unless otherwise noted. Room temperature is between about 20 and 25° C.

Method A:

Compounds of formula (I-A) wherein G is hydrogen, D is oxygen and Q is sulfur can be prepared by the reaction of N-alkylation reaction of 1,1,1-halogenalkyl-3-(2-thiazolidinylidene)-propanone of formula (III; n=0) with aryl or hetaryl halides of formula (II) as shown in Scheme 1.

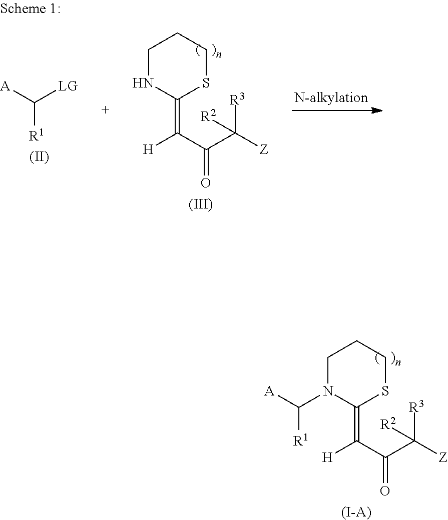

Typically, for the coupling reaction a metal salt such as caesium carbonate is used in the method of Scheme 1. The reaction can be carried out at temperatures ranging from room temperature to 40° C. A typical solvent for carrying out the process is N,N-dimethylformamide.

Compounds of formula (II) wherein A is hetaryl, R¹ is hydrogen or alkyl and LG is a Leaving Group such as halogen are well known in the art.

Compounds of formula (III) wherein R², R³ and Z is halogen(alkyl) and n is 0 can be prepared as described: 1,1,1-Trifluoro-3-(2-thiazolidinylidene)-2-propanone (R², R³, Z=F; n=0, DE 3639877 A1, 1988 WO 2012/029672 A1), 3,3,4,4,5,5,5-heptafluoro-1-(2-thiazolidinylidene)-2-pentanone, (R², R³=F; Z=CF₂CF₃; n=0, EP 652215 A1, 1995); 1-chloro-1,1-difluoro-3-(2-thiazolidinylidene)-2-propanone (R², R³=F; Z=Cl; n=0, EP 652215 A1, 1995); 1-chloro-1,1-difluoro-3-(tetrahydro-2H-1,3-thiazin-2-ylidene)-2-propanone (R², R³=F; Z=Cl; n=1; EP 652215 A1, 1995); 3,3,4,4,4-pentafluoro-1-(tetrahydro-2H-1,3-thiazin-2-ylidene)-2-butanone (R², R³=F; Z=CF₃; n=1; EP 652215 A1, 1995).

Method B:

Compounds of formula (I-B) (i.e. formula (I) wherein G is hydrogen, D is oxygen and Q is NH) can be prepared by the reaction of N¹-heraryl-methyl-1,2-ethanediamines of formula (IV) with 4,4-dialkoxy-1,1,1-trifluoro-3-buten-2-ones (prepared according to WO 2007/ 067836 A2) of formula (V) as shown in Scheme 2.

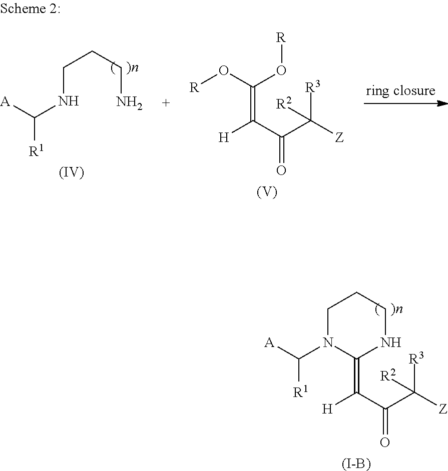

Typically, the ring closure reaction is carried out in acetonitrile as solvent. The reaction can be carried out at temperatures ranging from room temperature to the reflux temperature of the solvent.

Compounds of formula (IV) wherein A is hetaryl, R¹ is hydrogen and n is 0 are well known in the art or they can be prepared as described: N¹-[(6-fluoro-3-pyridinyl)methyl]-1,2-ethanediamine (A=6-fluoro-3-pyridinyl, R¹, R¹=H, n=0; EP 163855 A1, 1985), N¹-[(6-chloro-3-pyridinyl)methyl]-1,2-ethanediamine (A=6-chloro-3-pyridinyl, R¹=H, n=0; WO 2007/101369 A1), N¹-[(6-bromo-3-pyridinyl)methyl]-1,2-ethanediamine (A=6-bromo-3-pyridinyl, R¹=H, n=0; EP 163855 A1, 1985); N¹-[(6-fluoro-3-pyridinyl)methyl]-1,3-propanediamine (A=fluoro-3-pyridinyl;, R¹=H, n=1; EP 163855 A1, 1985), N¹-[(6-chloro-3-pyridinyl)methyl]-1,3-propanediamine (A=chloro-3-pyridinyl; R¹=H, n=1; EP 163855 A1, 1985), N¹-[(6-bromo-3-pyridinyl)methyl]-1,3-propanediamine (A=bromo-3-pyridinyl; R¹=H, n=1; EP 163855 A1, 1985).

For example, N¹-[(6-chloro-5-fluoro-3-pyridinyl)methyl]-1,2-ethanediamine (IVa) wherein A is (6-chloro-5-fluoro-3-pyridinyl)methyl, R¹ is hydrogen and n is 0 can be prepared by N¹-alkylation from ethylenediamine (VII) with 5-(bromomethyl)-2-chloro-3-fluoro-pyridine (VI) (prepared according to EP 2633756 A1, 2013) in acetonitrile as shown in Scheme 3 (see Example 1, step 1).

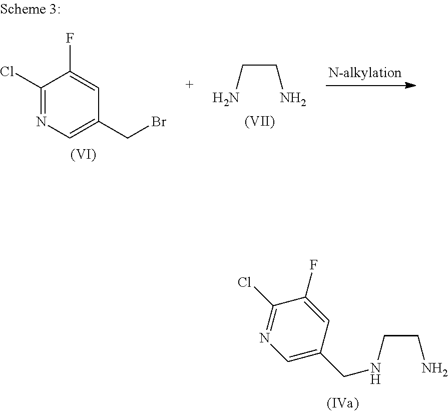

Compounds of formula (V) wherein R is alkyl and R², R³ and Z are halogen(alkyl) can be prepared as described: 1,1,1-trifluoro-4,4-dimethoxy-3-buten-2-one, (R=CH₃; R², R³, Z=F; M. Lubbe, et al. Synlett 2008, 12, 1862-1864); 1,1-diethoxy -4,4,5,5,5-pentafluoro-1-penten-3-one, (R=CH₂CH₃; R², R³=F; Z=CF₃; M. A. P. Martins, et al. J. Fluor. Chem. 2003, 123(2), 261-265); 1-chloro-1,1-difluoro-4, 4-dimethoxy-3-buten-2-one, (R=CH₃; R², R³=F; Z=Cl; S. Reimann, et al. J. Fluor. Chem. 2012, 139, 28-45); 1,1-difluoro-4,4-dimethoxy-3-buten-2-one, (R=CH₃; R², R³=F; Z=H; S. Reimann, et al. J. Fluor. Chem. 2012, 139, 28-45).

For example, the ring closure of compounds of formula (IV) such as N¹-[(6-chloro-5-fluoro-3-pyridinyl) methyl]-1,2-ethanediamine (IVa) and compounds of formula (V) such as 4,4-diethoxy-1,1,1-trifluoro-3-buten-2-one (prepared according to WO 2007/067836 A2) can be performed according to literature methods (for instance DE 3639877 A1, 1988). The ring closure can be preferably performed in a suitable solvent, for instance in acetonitrile at room temperature and reflux.

Method C:

Compounds of formula (I-D) (i.e. formula (I) wherein D is sulfur, G is hydrogen, and Q is sulfur or NH) can be prepared by thionation of the corresponding compounds of formula (I-C) (i.e. formula (I) wherein D is oxygen, G is hydrogen and Q is sulfur or NH) by treatment with an thionation reagent as shown in Scheme 4.

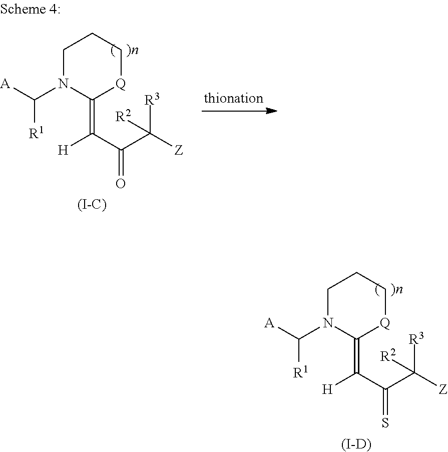

Compounds of formula (I-C) wherein the definitions of A, Q, Z, n, R¹, R² and R³ is as defined above in the Summary of the Invention can be prepared according to the reaction schemes 1 and 2.

Typically, an thionation reagent such as 2,4-bis(4-methoxyphenyl)-2,4-dithioxo-1,3,2,4-dithiadiphosphetane (“Lawesson's reagent”) or P2S₅ are used in the method of Scheme 3. The thionation step can be performed according to known literature methods (for instance WO 2016/016131 A1). The thionation can be preferably performed with P₂S₅ in the presence of a base, such as sodium carbonate in a suitable solvent, for instance in tetrahydrofurane. The thionation step can also be carried out using Lawesson's reagent in a suitable colvent, preferably in toluene.

The reactants ca be reacted in the presence of a base. Examples of suitable bases are alkyli metal or alkaline earth metal hydroxides, alkyli metal hydrides, alkali metal or alkaline earth metal amides, alkali metal or alkaline earth metal alkoxides, alkali metal or alkaline earth metal acetates, alkali metal or alkaline earth metal carbonates alkali metal or alkaline earth metal dialkylamides, alkali metal or alkaline earth metal alkylsilylamides, alkylamines, alkylendiamines, free or N-alkylated saturated or unsaturated cycloalkylamines, basic heterocycles, ammonium hydroxides and carboxyxlic amines, examples which may be mentioned are sodium hydroxide, sodium hydride, sodium amide, sodium methoxide, sodium acetate, sodium carbonate, potassium tert-butoxide, potassium hydroxide, potassium carbonate, potassium hydride, lithium diisopropylamide, potassium bis(trimethylsilyl)amide, calcium hydride, triethylamine, diisopropylethylamine, triethylendiamine, cyclohexylamine, N-cyclohexyl-N,N-dimethylamine, N,N-diethylaniline, pyridine, 4-(N,N-dimethylamino)pyridine, quinuclidine, N-methyl-morpholine, benzyltrimethylammonium hydroxide and 1,8-diazabicyclo [5.4.0] unde cen-7-ene (DBU).

The reactants can be reacted with each other as such, i.e. without adding a solvent or diluent. In most cases, however, it is advantageous to add an inert solvent or diluent or a mixture of these. If the reaction is carried out in the presence of a base, bases which are employed in excess, such as triethylamine, pyridine, N-methyl-morpholine or N,N-diethylaniline, may also act as dolvents or diluents.

The reaction is advantageous carried out in a temperature range from approximately -80° C. to approximately +140° C., preferably from approximately −30° C. to approximately +100° C., in many cases in the range between ambient temperature and approximately +80°.

Method D:

Compounds of formula (I-E) (i.e. formula (I) wherein D is oxygen, G is halogen and Q is sulfur or NH) can be prepared by electrophilc halogenation of the corresponding compounds of formula (I-C) (i.e. formula (I) wherein D is oxygen, G is hydrogen and Q is sulfur or NH) by treatment with an electrophilc halogenation reagent as shown in Scheme 5.

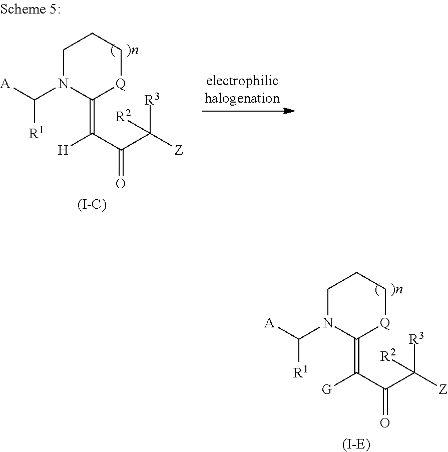

Compounds of formula (I-C) wherein the definitions of A, Q, Z, n, R¹, R² and R³ is as defined above in the Summary of the Invention can be prepared according to the reaction schemes 1 and 2.

Typically, an electrophilic halogenation reagent such as N-chlorosuccinimide (NCS), N-bromosuccinimide (NBS), N-iodosuccinimide (NIS) or electrophilic fluorination reagents like Selectfluor®, 1-fluoropyridinium triflate (Py-F⁺TFO⁻) or N-fluorobis(phenylsulfonyl)amine (NFSI) are used in the method of Scheme 1. The reaction can be performed in a solvent, for instance in dichloromethyne or acetonitrile (for instance WO 2016/016131 Al) at a temperature range of −10 to 100° C., preferably beween 0 and 60° C.

A compound of formula (I) can be converted in a manner known per se into another compound of formula (I) by replacing one or more substituents of the strating compound fo formula (I) in the customary manner by (an)other substituent(s) according to the invention.

Depending on the choice of the reaction conditions and starting materials which are suitable in each case, it is possible, for example, in one reaction step only to replace one substituent by another substituent according to the invention, or a plurality of substituents can be replaced by other substituents according to the invention in the same reaction step.

Salts of compounds of the formula (I) can be prepared in a manner known per se. Thus, for example acid addition salts of compounds of formula (I) are obtained by treatment with a suitable acid or a suitable ion exchanger reagent and salts with bases are obtained by treatment with a suitable base or with a suitable ion exchanger reagent.

Salts of compounds for formula (I) can be converted in the costomary manner into free compounds (I), acid addition salts, for example, by treatment with a suitable basic compound or with a suitable ion exchanger reagent.

Salts of compounds of formula (I) can be converted in a manner known per se into other salts of compounds of formula (I), acid addition salts, for example, into other acid addition salts, for example by treatment of a salt of inorganic acid such as hydrochloride with a sutable metal salt such as sodium, barium or silver salt, in a suitable solvent in which an inorganic salt which forms, is insoluble and thus precipitates from the reaction mixture.

Depending on the procedure or reaction conditions, the compounds of formula (I), which have salt-forming properties can be obtained in free form or in the form of salts.

The compounds of the formula (I) and, where appropriate, the tautomers therefo, in each case in free form or in salt form, can be present in the form of one of the isomers which are possible or as mixture of these, for example in form of pure isomers, such as antipodes and/or diastereomers, or as isomer mixtures, such as enantiomer mixtures, for example racemates, diastereomer mixtures or racemate mixtures, depending on the number, absolute and relative configuration of asymmetric carbon atoms which occur in the molecule and/or depending on the configuration of of non-aromatic double bonds which occur in the molecule; the invention relates to the pure isomers and also to all isomer mixtures which are possible and is to be understood in each case in this sense hereinabove and hereinbelow, even when stereochmical details are nocht mentioned specifically in each case.

Diastereomer mixtures or racemate mixtures of compounds of formula (I), in free form or in salt form, which can be obtained depending on which starting materials and procedures have been chosen can be separated in a known manner into the pure diastereomers or racemates on the basis of the physicochemical differences of the components, for example by fractional crystallization, desillation and/or chromatography.

Enantiomer mixtures, such as racemates, which can be obtained in a similar manner can be resolved into the optical antipodes by known methods, for example by recrystallization from an optically active solvent, by chromatography on chiral adsorbents, for example high-performance liquid chropatography (HPLC) on acetyl cellulose, with the aid of suitable microorganisms, by cleavage with specific, immobilized enzymes, via the formation of inclusion compounds, for example using chiral crown ethers, where only one enantiomer is complexed, or by conversion into diastereomeric salts, for example by reacting a basic end-product racemate with an optically active acid, such as carboxylic acid, for example camphor, tartaric or malic acid, or sulfonic acid, for example camphorsulfonic acid, and separating the diastereomer mixture which can be obtained in this manner, for example by fractional crystallization based on their differing solubilities, to give diastereomers, from which the desired enantiomer can be set free by the action of suitable agents, for example basic agents.

Pure diastereomers or enantiomers can be obtained according to the invention not only by separating suitable isomer mixtures, but alsi by generally known methods of diastereoselective or enantioselective synthesis, for example by carrying out the process according to the invention with starting materials of a suitable stereochemistry. N-oxides can be prepared by reaction a compound of formula (I) with a suitable oxidizing reagent, for example the H2O₂/urea adduct in the presence of an acid anhydride, e.g. trifluoroacetc anhydride. Such oxidations are known from the literature (see WO 2000/15615 A1).

It is advantageous to isolate or synthesize in each case the biologically more effective isomer, for example enantiomer or diastereomer, or isomer mixture, for example enantiomer mixture or diastereomer mixture, if the individual components have a different biological activity.

The compounds of formula (I) and, where appropriate, the tautomers thereof, in each case in free form or in salt form, can, if appropriate, also be obtained in the form of hydrates and/or include other solvents, for example those which may have been used for crystallization of compounds which are present in solid form.

The active compounds according to the invention, in combination with good plant tolerance and favourable toxicity to warm-blooded animals and being tolerated well by the environment, are suitable for protecting plants and plant organs, for increasing the harvest yields, for improving the quality of the harvested material and for controlling animal pests, in particular insects, arachnids, helminths, nematodes and molluscs, which are encountered in agriculture, in horticulture, in animal husbandry, in forests, in gardens and leisure facilities, in the protection of stored products and of materials, and in the hygiene sector. They may be preferably employed as plant protection agents. They are active against normally sensitive and resistant species and against all or some stages of development. The abovementioned pests include:

From the order of the Anoplura (Phthiraptera), for example, Damalinia spp., Haematopinus spp., Linognathus spp., Pediculus spp., Trichodectes spp.

From the class of the Arachnida, for example, Acarus siro, Aceria sheldoni, Aculops spp., Aculus spp., Amblyomma spp., Argas spp., Boophilus spp., Brevipalpus spp., Bryobia praetiosa, Chorioptes spp., Dermanyssus gallinae, Eotetranychus spp., Epitrimerus pyri, Eutetranychus spp., Eriophyes spp., Hemitarsonemus spp., Hyalomma spp., Ixodes spp., Latrodectus mactans, Metatetranychus spp., Oligonychus spp., Ornithodoros spp., Panonychus spp., Phyllocoptruta oleivora, Polyphagotarsonemus latus, Psoroptes spp., Rhipicephalus spp., Rhizoglyphus spp., Sarcoptes spp., Scorpio maurus, Stenotarsonemus spp., Tarsonemus spp., Tetranychus spp., Vasates lycopersici.

From the class of the Bivalva, for example, Dreissena spp.

From the order of the Chilopoda, for example, Geophilus spp., Scutigera spp.

From the order of the Coleoptera, for example, Acanthoscelides obtectus, Adoretus spp., Agelastica alni, Agriotes spp., Amphimallon solstitialis, Anobium punctatum, Anoplophora spp., Anthonomus spp., Anthrenus spp., Apogonia spp., Atomaria spp., Attagenus spp., Bruchidius obtectus, Bruchus spp., Ceuthorhynchus spp., Cleonus mendicus, Conoderus spp., Cosmopolites spp., Costelytra zealandica, Curculio spp., Cryptorhynchus lapathi, Dermestes spp., Diabrotica spp., Epilachna spp., Faustinus cubae, Gibbium psylloides, Heteronychus arator, Hylamorpha elegans, Hylotrupes bajulus, Hypera postica, Hypothenemus spp., Lachnosterna consanguinea, Leptinotarsa decemlineata, Lissorhoptrus oryzophilus, Lixus spp., Lyctus spp., Meligethes aeneus, Melolontha melolontha, Migdolus spp., Monochamus spp., Naupactus xanthographus, Niptus hololeucus, Oryctes rhinoceros, Oryzaephilus surinamensis, Otiorrhynchus sulcatus, Oxycetonia jucunda, Phaedon cochleariae, Phyllophaga spp., Popillia japonica, Premnotrypes spp., Psylliodes chrysocephala, Ptinus spp., Rhizobius ventralis, Rhizopertha dominica, Sitophilus spp., Sphenophorus spp., Sternechus spp., Symphyletes spp., Tenebrio molitor, Tribolium spp., Trogoderma spp., Tychius spp., Xylotrechus spp., Zabrus spp.

From the order of the Collembola, for example, Onychiurus armatus.

From the order of the Dermaptera, for example, Forficula auricularia.

From the order of the Diplopoda, for example, Blaniulus guttulatus.

From the order of the Diptera, for example, Aedes spp., Anopheles spp., Bibio hortulanus, Calliphora erythrocephala, Ceratitis capitata, Chrysomyia spp., Cochliomyia spp., Cordylobia anthropophaga, Culex spp., Cuterebra spp., Dacus oleae, Dermatobia hominis, Drosophila spp., Fannia spp., Gastrophilus spp., Hylemyia spp., Hyppobosca spp., Hypoderma spp., Liriomyza spp., Lucilia spp., Musca spp., Nezara spp., Oestrus spp., Oscinella frit, Pegomyia hyoscyami, Phorbia spp., Stomoxys spp., Tabanus spp., Tannia spp., Tipula paludosa, Wohlfahrtia spp.

From the class of the Gastropoda, for example, Anion spp., Biomphalaria spp., Bulinus spp., Deroceras spp., Galba spp., Lymnaea spp., Oncomelania spp., Succinea spp.

From the class of the Helminths, for example, Ancylostoma duodenale, Ancylostoma ceylanicum, Acylostoma braziliensis, Ancylostoma spp., Ascaris lubricoides, Ascaris spp., Brugia malayi, Brugia timori, Bunostomum spp., Chabertia spp., Clonorchis spp., Cooperia spp., Dicrocoelium spp, Dictyocaulus filaria, Diphyllobothrium latum, Dracunculus medinensis, Echinococcus granulosus, Echinococcus multilocularis, Enterobius vermicularis, Faciola spp., Haemonchus spp., Heterakis spp., Hymenolepis nana, Hyostrongulus spp., Loa Loa, Nematodirus spp., Oesophagostomum spp., Opisthorchis spp., Onchocerca volvulus, Ostertagia spp., Paragonimus spp., Schistosomen spp., Strongyloides fuelleborni, Strongyloides stercoralis, Stronyloides spp., Taenia saginata, Taenia solium, Trichinella spiralis, Trichinella nativa, Trichinella britovi, Trichinella nelsoni, Trichinella pseudopsiralis, Trichostrongulus spp., Trichuris trichuria, Wuchereria bancrofti.

It is furthermore possible to control protozoa, such as Eimeria.

From the order of the Heteroptera, for example, Anasa tristis, Antestiopsis spp., Blissus spp., Calocoris spp., Campylomma livida, Cavelerius spp., Cimex spp., Creontiades dilutus, Dasynus piperis, Dichelops furcatus, Diconocoris hewetti, Dysdercus spp., Euschistus spp., Eurygaster spp., Heliopeltis spp., Horcias nobilellus, Leptocorisa spp., Leptoglossus phyllopus, Lygus spp., Macropes excavatus, Miridae, Nezara spp., Oebalus spp., Pentomidae, Piesma quadrata, Piezodorus spp., Psallus seriatus, Pseudacysta persea, Rhodnius spp., Sahlbergella singularis, Scotinophora spp., Stephanitis nashi, Tibraca spp., Triatoma spp.

From the order of the Homoptera, for example, Acyrthosipon spp., Aeneolamia spp., Agonoscena spp., Aleurodes spp., Aleurolobus barodensis, Aleurothrixus spp., Amrasca spp., Anuraphis cardui, Aonidiella spp., Aphanostigma pini, Aphis spp., Arboridia apicalis, Aspidiella spp., Aspidiotus spp., Atanus spp., Aulacorthum solani, Bemisia spp., Brachycaudus helichrysii, Brachycolus spp., Brevicoryne brassicae, Calligypona marginata, Carneocephala fulgida, Ceratovacuna lanigera, Cercopidae, Ceroplastes spp., Chaetosiphon fragaefolii, Chionaspis tegalensis, Chlorita onukii, Chromaphis juglandicola, Chrysomphalus ficus, Cicadulina mbila, Coccomytilus halli, Coccus spp., Cryptomyzus ribis, Dalbulus spp., Dialeurodes spp., Diaphorina spp., Diaspis spp., Doralis spp., Drosicha spp., Dysaphis spp., Dysmicoccus spp., Empoasca spp., Eriosoma spp., Erythroneura spp., Euscelis bilobatus, Geococcus coffeae, Homalodisca coagulata, Hyalopterus arundinis, Icerya spp., Idiocerus spp., Idioscopus spp., Laodelphax striatellus, Lecanium spp., Lepidosaphes spp., Lipaphis erysimi, Macrosiphum spp., Mahanarva fimbriolata, Melanaphis sacchari, Metcalfiella spp., Metopolophium dirhodum, Monellia costalis, Monelliopsis pecanis, Myzus spp., Nasonovia ribisnigri, Nephotettix spp., Nilaparvata lugens, Oncometopia spp., Orthezia praelonga, Parabemisia myricae, Paratrioza spp., Parlatoria spp., Pemphigus spp., Peregrinus maidis, Phenacoccus spp., Phloeomyzus passerinii, Phorodon humuli, Phylloxera spp., Pinnaspis aspidistrae, Planococcus spp., Protopulvinaria pyriformis, Pseudaulacaspis pentagona, Pseudococcus spp., Psylla spp., Pteromalus spp., Pyrilla spp., Quadraspidiotus spp., Quesada gigas, Rastrococcus spp., Rhopalosiphum spp., Saissetia spp., Scaphoides titanus, Schizaphis graminum, Selenaspidus articulatus, Sogata spp., Sogatella furcifera, Sogatodes spp., Stictocephala festina, Tenalaphara malayensis, Tinocallis caryaefoliae, Tomaspis spp., Toxoptera spp., Trialeurodes vaporariorum, Trioza spp., Typhlocyba spp., Unaspis spp., Viteus vitifolii.

From the order of the Hymenoptera, for example, Diprion spp., Hoplocampa spp., Lasius spp., Mono- morium pharaonis, Vespa spp.

From the order of the Isopoda, for example, Armadillidium vulgare, Oniscus asellus, Porcellio scaber.

From the order of the Isoptera, for example, Reticulitermes spp., Odontotermes spp.

From the order of the Lepidoptera, for example, Acronicta major, Aedia leucomelas, Agrotis spp., Alabama argillacea, Anticarsia spp., Barathra brassicae, Bucculatrix thurberiella, Bupalus piniarius, Cacoecia podana, Capua reticulana, Carpocapsa pomonella, Cheimatobia brumata, Chilo spp., Choristoneura fumiferana, Clysia ambiguella, Cnaphalocerus spp., Earias insulana, Ephestia kuehniella, Euproctis chrysorrhoea, Euxoa spp., Feltia spp., Galleria mellonella, Helicoverpa spp., Heliothis spp., Hofmannophila pseudospretella, Homona magnanima, Hyponomeuta padella, Laphygma spp., Lithocolletis blancardella, Lithophane antennata, Loxagrotis albicosta, Lymantria spp., Malacosoma neustria, Mamestra brassicae, Mocis repanda, Mythimna separata, Oria spp., Oulema oryzae, Panolis flammea, Pectinophora gossypiella, Phyllocnistis citrella, Pieris spp., Plutella xylostella, Prodenia spp., Pseudaletia spp., Pseudoplusia includens, Pyrausta nubilalis, Spodoptera spp., Thermesia gemmatalis, Tinea pellionella, Tineola bisselliella, Tortrix viridana, Trichoplusia spp.

From the order of the Orthoptera, for example, Acheta domesticus, Blatta orientalis, Blattella germanica, Gryllotalpa spp., Leucophaea maderae, Locusta spp., Melanoplus spp., Periplaneta americana, Schistocerca gregaria.

From the order of the Siphonaptera, for example, Ceratophyllus spp., Xenopsylla cheopis.

From the order of the Symphyla, for example, Scutigerella immaculata.

From the order of the Thysanoptera, for example, Baliothrips biformis, Enneothrips flavens, Frankliniella spp., Heliothrips spp., Hercinothrips femoralis, Kakothrips spp., Rhipiphorothrips cruentatus, Scirtothrips spp., Taeniothrips cardamoni, Thrips spp.

From the order of the Thy sanura, for example, Lepisma saccharina.

The phytoparasitic nematodes include, for example, Anguina spp., Aphelenchoides spp., Belonoaimus spp., Bursaphelenchus spp., Ditylenchus dipsaci, Globodera spp., Heliocotylenchus spp., Heterodera spp., Longidorus spp., Meloidogyne spp., Pratylenchus spp., Radopholus similis, Rotylenchus spp., Trichodorus spp., Tylenchorhynchus spp., Tylenchulus spp., Tylenchulus semipenetrans, Xiphinema spp.

If appropriate, the compounds according to the invention can, at certain concentrations or application rates, also be used as herbicides, safeners, growth regulators or agents to improve plant properties, or as microbicides, for example as fungicides, antimycotics, bactericides, viricides (including agents against viroids) or as agents against MLO (Mycoplasma-like organisms) and RLO (Rickettsia-like organisms). If appropriate, they can also be employed as intermediates or precursors for the synthesis of other active compounds.

The active compounds can be converted to the customary formulations, such as solutions, emulsions, wettable powders, water- and oil-based suspensions, powders, dusts, pastes, soluble powders, soluble granules, granules for broadcasting, suspension-emulsion concentrates, natural materials impregnated with active compound, synthetic materials impregnated with active compound, fertilizers and microencapsulations in polymeric substances.

These formulations are produced in a known manner, for example by mixing the active compounds with extenders, that is liquid solvents and/or solid carriers, optionally with the use of surfactants, that is emulsifiers and/or dispersants and/or foam-formers. The formulations are prepared either in suitable plants or else before or during the application.

Suitable for use as auxiliaries are substances which are suitable for imparting to the composition itself and/or to preparations derived therefrom (for example spray liquors, seed dressings) particular properties such as certain technical properties and/or also particular biological properties. Typical suitable auxiliaries are: extenders, solvents and carriers.

Suitable extenders are, for example, water, polar and nonpolar organic chemical liquids, for example from the classes of the aromatic and non-aromatic hydrocarbons (such as paraffins, alkylbenzenes, alkylnaphthalenes, chlorobenzenes), the alcohols and polyols (which, if appropriate, may also be substituted, etherified and/or esterified), the ketones (such as acetone, cyclohexanone), esters (including fats and oils) and (poly)ethers, the unsubstituted and substituted amines, amides, lactams (such as N-alkylpyrrolidones) and lactones, the sulphones and sulphoxides (such as dimethyl sulphoxide).

If the extender used is water, it is also possible to employ, for example, organic solvents as auxiliary solvents. Essentially, suitable liquid solvents are: aromatics, such as xylene, toluene or alkylnaphthalenes, chlorinated aromatics and chlorinated aliphatic hydrocarbons such as chlorobenzenes, chloroethylenes or methylene chloride, aliphatic hydrocarbons such as cyclohexane or paraffms, for example petroleum fractions, mineral and vegetable oils, alcohols such as butanol or glycol and also their ethers and esters, ketones such as acetone, methyl ethyl ketone, methyl isobutyl ketone or cyclohexanone, strongly polar solvents such as dimethyl sulphoxide, and also water.

Suitable Solid Carriers are:

for example, ammonium salts and ground natural minerals such as kaolins, clays, talc, chalk, quartz, attapulgite, montmorillonite or diatomaceous earth, and ground synthetic minerals, such as finely divided silica, alumina and silicates; suitable solid carriers for granules are: for example, crushed and fractionated natural rocks such as calcite, marble, pumice, sepiolite and dolomite, and also synthetic granules of inorganic and organic meals, and granules of organic material such as paper, sawdust, coconut shells, maize cobs and tobacco stalks; suitable emulsifiers and/or foam-formers are: for example, nonionic and anionic emulsifiers, such as polyoxyethylene fatty acid esters, polyoxyethylene fatty alcohol ethers, for example alkylaryl polyglycol ethers, alkylsulphonates, alkyl sulphates, arylsulphonates and also protein hydrolysates; suitable dispersants are nonionic and/or ionic substances, for example from the classes of the alcohol-POE- and/or -POP-ethers, acid and/or POP-POE esters, alkyl aryl and/or POP-POE ethers, fat- and/or POP-POE adducts, POE- and/or POP-polyol derivatives, POE- and/or POP-sorbitan or -sugar adducts, alkyl or aryl sulphates, alkyl- or arylsulphonates and alkyl or aryl phosphates or the corresponding PO-ether adducts. Furthermore, suitable oligo- or polymers, for example those derived from vinylic monomers, from acrylic acid, from EO and/or PO alone or in combination with, for example, (poly)alcohols or (poly)amines It is also possible to employ lignin and its sulphonic acid derivatives, unmodified and modified celluloses, aromatic and/or aliphatic sulphonic acids and their adducts with formaldehyde.

Tackifiers such as carboxymethylcellulose and natural and synthetic polymers in the form of powders, granules or latices, such as gum arabic, polyvinyl alcohol and polyvinyl acetate, as well as natural phospholipids such as cephalins and lecithins, and synthetic phospholipids, can be used in the formulations.

It is possible to use colorants such as inorganic pigments, for example iron oxide, titanium oxide and Prussian Blue, and organic dyestuffs, such as alizarin dyestuffs, azo dyestuffs and metal phthalocyanine dyestuffs, and trace nutrients such as salts of iron, manganese, boron, copper, cobalt, molybdenum and zinc.

Other possible additives are perfumes, mineral or vegetable, optionally modified oils, waxes and nutrients (including trace nutrients), such as salts of iron, manganese, boron, copper, cobalt, molybdenum and zinc.

Stabilizers, such as low-temperature stabilizers, preservatives, antioxidants, light stabilizers or other agents which improve chemical and/or physical stability may also be present.

The formulations generally comprise between 0.01 and 98% by weight of active compound, preferably between 0.5 and 90%.

The active compound according to the invention can be used in its commercially available formulations and in the use forms, prepared from these formulations, as a mixture with other active compounds, such as insecticides, attractants, sterilizing agents, bactericides, acaricides, nematicides, fungicides, growth-regulating substances, herbicides, safeners, fertilizers or semiochemicals.

Particularly favourable mixing components are, for example, the following compounds:

Fungicides:

Inhibitors of nucleic acid synthesis
benalaxyl, benalaxyl-M, bupirimate, chiralaxyl, clozylacon, dimethirimol, ethirimol, furalaxyl, hymexazol, metalaxyl, metalaxyl-M, ofurace, oxadixyl, oxolinic acid

Inhibitors of mitosis and cell division
benomyl, carbendazim, diethofencarb, fuberidazole, pencycuron, thiabendazole, thiophanat-methyl, zoxamide

Inhibitors of respiratory chain complex I
diflumetorim

Inhibitors of respiratory chain complex II
boscalid, carboxin, fenfuram, flutolanil, furametpyr, mepronil, oxycarboxin, penthiopyrad, thifluzamide

Inhibitors of respiratory chain complex III
azoxystrobin, cyazofamid, dimoxystrobin, enestrobin, famoxadone, fenamidone, fluoxastrobin, kresoxim-methyl, metominostrobin, orysastrobin, pyraclostrobin, picoxystrobin, trifloxystrobin

Decouplers
dinocap, fluazinam

Inhibitors of ATP production
fentin acetate, fentin chloride, fentin hydroxide, silthiofam

Inhibitors of amino acid biosynthesis and protein biosynthesis
andoprim, blasticidin-S, cyprodinil, kasugamycin, kasugamycin hydrochloride hydrate, mepanipyrim, pyrimethanil

Inhibitors of signal transduction
fenpiclonil, fludioxonil, quinoxyfen

Inhibitors of lipid and membrane synthesis
chlozolinate, iprodione, procymidone, vinclozolin
ampropylfos, potassium-ampropylfos, edifenphos, iprobenfos (IBP), isoprothiolane, pyrazophos
tolclofos-methyl, biphenyl
iodocarb, propamocarb, propamocarb hydrochloride

Inhibitors of ergosterol biosynthesis
fenhexamid,
azaconazole, bitertanol, bromuconazole, cyproconazole, diclobutrazole, difenoconazole, diniconazole, diniconazole-M, epoxiconazole, etaconazole, fenbuconazole, fluquinconazole, flusilazole, flutriafol, furconazole, furconazole-cis, hexaconazole, imibenconazole, ipconazole, metconazole, my clobutanil, paclobutrazole, penconazole, propiconazole, prothioconazole, simeconazole, tebuconazole, tetraconazole, triadimefon, triadimenol, triticonazole, uniconazole, voriconazole, imazalil, imazalil sulphate, oxpoconazole, fenarimol, flurprimidole, nuarimol, pyrifenox, triforine, pefurazoate, prochloraz, triflumizole, viniconazole,
aldimorph, dodemorph, dodemorph acetate, fenpropimorph, tridemorph, fenpropidin, spiroxamine,
naftifine, pyributicarb, terbinafine

Inhibitors of cell wall synthesis
benthiavalicarb, bialaphos, dimethomorph, flumorph, iprovalicarb, polyoxins, polyoxorim, validamycin A

Inhibitors of melanin biosynthesis
capropamid, diclocymet, fenoxanil, phthalid, pyroquilon, tricyclazole

Resistance inductors
acibenzolar-S-methyl, probenazole, tiadinil

Multisite
captafol, captan, chlorothalonil, copper salts such as: copper hydroxide, copper naphthenate, copper oxychloride, copper sulphate, copper oxide, oxine-copper and Bordeaux mixture, dichlofluanid, dithianon, dodine, dodine free base, ferbam, folpet, fluorofolpet, guazatine, guazatine acetate, iminoctadine, iminoctadine albesilate, iminoctadine triacetate, mancopper, mancozeb, maneb, metiram, metiram zinc, propineb, sulphur and sulphur preparations containing calcium polysulphide, thiram, tolylfluanid, zineb, ziram

Unknown mechanism
amibromdol, benthiazol, bethoxazin, capsimycin, carvone, chinomethionat, chloropicrin, cufraneb, cyflufenamid, cymoxanil, dazomet, debacarb, diclomezine, dichlorophen, dicloran, difenzoquat, difenzoquat methyl sulphate, diphenylamine, ethaboxam, ferimzone, flumetover, flusulphamide, fluopicolide, fluoroimide, hexachlorobenzene, 8-hydroxyquinoline sulphate, irumamycin, methasulfocarb, metrafenone, methyl isothiocyanate, mildiomycin, natamycin, nickel dimethyl dithiocarbamate, nitrothal-isopropyl, octhilinone, oxamocarb, oxyfenthiin, pentachlorophenol and salts, 2-phenylphenol and salts, piperalin, propanosine-sodium, proquinazid, pyrrol nitrin, quintozene, tecloftalam, tecnazene, triazoxide, trichlamide, zarilamid and 2,3,5,6-tetrachloro-4-(methylsulphonyl)pyridine, N-(4-chloro-2-nitrophenyl)-N-ethyl-4-methylbenzenesulphonamide, 2-amino-4-methyl-N-phenyl-5-thiazolec arboxamide, 2-chloro-N-(2,3-dihydro-1,1,3-trimethyl-1H-inden-4-yl)-3-pyridinecarboxamide, 3 45-(4-chlorophenyl)-2,3-dimethylisoxazolidin-3-yl]pyridine, cis-1-(4-chlorophenyl)-2-(1H-1,2,4-triazol-1-yl)cycloheptanol, 2,4-dihydro-5-methoxy -2-methyl-4-[[[[1- [3-(trifluoromethy Ophenyl]methy lidene]amino]oxy]methyl]phenyl]-3H-1,2,3-triazol-3-one (185336-79-2), methyl 1-(2,3-dihydro-2,2-dimethyl-1H-inden-1-yl)-1H-imidazole -5-c arboxy late, 3,4,5-trichloro-2,6-pyridinedicarbonitrile, methyl 2- [ [[cyclopropyl[(4-methoxy-phenypimino]methyl]thio]methyl]-alpha-(methoxymethylene)benzacetate, 4-chloro-alpha-propynyloxy-N-[2-[3-methoxy -4-(2-propynyloxy)phenyl]ethyl]benzacetamide, (2 S)-N-[2-[4-[[3-(4-chlorophenyl)-2-propynyl]oxy]-3-methoxy phenyl]ethyl]-3-methyl-2-[(methylsulphon-yl)amino]butanamide, 5-chloro-7-(4-methylpiperidin-1-yl)-6-(2,4,6-trifluorophenyl) [1,2,4]-triazolo[1,5-a]pyrimidine, 5-chloro-6-(2,4,6-trifluorophenyl)-N-[(1R)-1,2,2-trimethylpropyl]-[1,2,4]triazolo[1,5-a]pyrimidin-7-amine, 5-chloro-N-[(1R)-1,2-dimethy 1propyl]-6-(2,4,6-trifluorophenyl)[1,2,4]triazolo[1,5-a]pyrimidin-7-amine, N-[1-(5-bromo-3-chloropyridin-2-yl)ethyl]-2,4-dichloronicotinamide, N-(5-bromo-3-chloropyridin-2-yl)methyl-2,4-dichloro-nicotinamide, 2-butoxy-6-iodo-3-propylbenzopyranon-4-one, N-{(Z)-[(cyclopropylmethoxy)-imino][6-(difluoromethoxy)-2,3-difluorophenyl]methyl}-2-benzacetamide, N-(3-ethyl-3,5,5-trimethylcyclohexyl)-3-formylamino-2-hydroxybenzamide, 2-[[[[1-[3 (1-fluoro-2-phenyl-ethyl)oxy[phenyl]ethylidene]amino]oxy]methyl]-alpha-(methoxyimino)-N-methyl-alphaE-benzacetamide, N-{2-[3-chloro-5-(trifluoromethyl)pyridin-2-yl- ethyl}-2-(trifluoro-methyl)benzamide, N-(3′,4′-dichloro-5-fluorobiphenyl-2-yl)-3-(difluoromethyl)-1-methyl-1H-pyrazole-4-carboxamide, N-(6-methoxy-3-pyridinyl)cyclopropanecarboxamide, 1-[(4-methoxyphenoxy)methyl]-2,2-dimethylpropyl-1H-imidazole-1-carboxylic acid, O-1-[(4-methoxyphenoxy)methyl]-2,2-dimethylpropyl]-1H-imidazole-l-carbothioic acid, 2-(2-{[6-(3-chloro-2-methylphenoxy)-5-fluoropyrimidin-4-yl]oxy}phenyl)-2-(methoxy imino)-N-methyl-acetamide

Bactericides:

bronopol, dichlorophen, nitrapyrin, nickel dimethyldithiocarbamate, kasugamycin, octhilinone, furancarboxylic acid, oxytetracycline, probenazole, streptomycin, tecloftalam, copper sulphate and other copper preparations.

Insecticides/acaricides/nematicides:

Acetylcholine esterase (AChE) inhibitors
carbamates,
for example alanycarb, aldicarb, aldoxycarb, allyxycarb, aminocarb, bendiocarb, benfuracarb, bufencarb, butacarb, butocarboxim, butoxycarboxim, carbaryl, carbofuran, carbosulphan, cloethocarb, dimetilan, ethiofencarb, fenobucarb, fenothiocarb, formetanate, furathiocarb, isoprocarb, metam-sodium, methiocarb, methomyl, metolcarb, oxamyl, pirimicarb, promecarb, propoxur, thiodicarb, thiofanox, trimethacarb, XMC, xylylcarb, triazamate
organophosphates,
for example acephate, azamethiphos, azinphos (-methyl, -ethyl), bromophos-ethyl, bromfenvinfos (-methyl), butathiofos, cadusafos, carbophenothion, chlorethoxyfos, chlorfenvinphos, chlormephos, chlorpyrifos (-methyl/-ethyl), coumaphos, cyanofenphos, cyanophos, chlorfenvinphos, demeton-S-methyl, demeton-S-methylsulphone, dialifos, diazinon, dichlofenthion, dichlorvos/DDVP, dicrotophos, dimethoate, dimethylvinphos, dioxabenzofos, disulphoton, EPN, ethion, ethoprophos, etrimfos, famphur, fenamiphos, fenitrothion, fensulfothion, fenthion, flupyrazofos, fonofos, formothion, fosmethilan, fosthiazate, heptenophos, iodofenphos, iprobenfos, isazofos, isofenphos, isopropyl 0-salicylate, isoxathion, malathion, mecarbam, methacrifos, methamidophos, methidathion, mevinphos, monocrotophos, naled, omethoate, oxydemeton-methyl, parathion (-methyl/-ethyl), phenthoate, phorate, phosalone, phosmet, phosphamidon, phosphocarb, phoxim, pirimiphos (-methyl/-ethyl), profenofos, propaphos, propetamphos, prothiofos, prothoate, pyraclofos, pyridaphenthion, pyridathion, quinalphos, sebufos, sulfotep, sulprofos, tebupirimfos, temephos, terbufos, tetrachlorvinphos, thiometon, triazophos, triclorfon, vamidothion

Sodium channel modulators / voltage-dependent sodium channel blockers
pyrethroids,
for example acrinathrin, allethrin (d-cis-trans, d-trans), beta-cyfluthrin, bifenthrin, bioallethrin, bioallethrin-S-cyclopentyl isomer, bioethanomethrin, biopermethrin, bioresmethrin, chlovaporthrin, cis-cypermethrin, cis-resmethrin, cis-permethrin, clocythrin, cycloprothrin, cyfluthrin, cyhalothrin, cypermethrin (alpha-, beta-, theta-, zeta-), cyphenothrin, deltamethrin, empenthrin (1R isomer), esfenvalerate, etofenprox, fenfluthrin, fenpropathrin, fenpyrithrin, fenvalerate, flubrocythrinate, flucythrinate, flufenprox, flumethrin, fluvalinate, fubfenprox, gamma-cyhalothrin, imiprothrin, kadethrin, lambda-cyhalothrin, metofluthrin, permethrin (cis-, trans-), phenothrin (1R trans-isomer), prallethrin, profluthrin, protrifenbute, pyresmethrin, resmethrin, RU 15525, silafluofen, tau-fluvalinate, tefluthrin, terallethrin, tetramethrin (1R isomer), tralomethrin, transfluthrin, ZXI 8901, pyrethrins (pyrethrum)
DDT
oxadiazines,
for example indoxacarb
semicarbazones,
for example metaflumizone (BAS3201)

Acetylcholine receptor agonists/antagonists
chloronicotinyls,
for example acetamiprid, clothianidin, dinotefuran, imidacloprid, nitenpyram, nithiazine, thiacloprid, imidaclothiz, AKD-1022, thiamethoxam
nicotine, bensultap, cartap

Acetylcholine receptor modulators
spinosyns,
for example spinosad, spinetoram (XDE-175)

GABA-controlled chloride channel antagonists
organochlorines,
for example camphechlor, chlordane, endosulfan, gamma-HCH, HCH, heptachlor, lindane, methoxychlor
fiprols,
for example acetoprole, ethiprole, fipronil, pyrafluprole, pyriprole, vaniliprole

Chloride channel activators
mectins,
for example abamectin, emamectin, emamectin benzoate, ivermectin, lepimectin, milbemycin

Juvenile hormone mimetics,
for example diofenolan, epofenonane, fenoxycarb, hydroprene, kinoprene, methoprene, pyriproxifen, triprene

Ecdy sone agonists/disruptors
diacylhydrazines,
for example chromafenozide, halofenozide, methoxyfenozide, tebufenozide

Chitin biosynthesis inhibitors
benzoylureas,
for example bistrifluron, chlofluazuron, diflubenzuron, fluazuron, flucycloxuron, flufenoxuron, hexaflumuron, lufenuron, novaluron, noviflumuron, penfluron, teflubenzuron, triflumuron
buprofezin
cyromazine

Oxidative phosphorylation inhibitors, ATP disruptors
diafenthiuron
organotin compounds,
for example azocyclotin, cyhexatin, fenbutatin oxide

Oxidative phosphorylation decouplers acting by interrupting the H-proton gradient
pyrroles,
for example chlorfenapyr
dinitrophenols,
for example binapacyrl, dinobuton, dinocap, DNOC

Site-I electron transport inhibitors
METIs,
for example fenazaquin, fenpyroximate, pyrimidifen, pyridaben, tebufenpyrad, tolfenpyrad
hydramethylnon
dicofol

Site-II electron transport inhibitors
rotenone

Site-III electron transport inhibitors
acequinocyl, fluacrypyrim

Microbial disruptors of the insect gut membrane
Bacillus thuringiensis strains

Lipid synthesis inhibitors
tetronic acids,
for example spirodiclofen, spiromesifen
tetramic acids,
for example spirotetramat
carboxamides,
for example flonicamid
octopaminergic agonists,
for example amitraz

Inhibitors of magnesium-stimulated ATPase,
propargite
nereistoxin analogues,
for example thiocyclam hydrogen oxalate, thiosultap-sodium

Ryanodin receptor agonists
benzoic acid dicarboxamides,
for example flubendiamid
anthranilamides,
for example rynaxypyr (3-bromo-N-{4-chloro-2-methyl-6-1[(methylamino)carbonyl]phenyl}1-1-(3-chloropyridin-2-yl)-1H-pyrazole-5-carboxamide)

Biologicals, hormones or pheromones
azadirachtin, Bacillus spec., Beauveria spec., codlemone, Metarrhizium spec., Paecilomyces spec., thuringiensin, Verticillium spec.

Active compounds with unknown or unspecific mechanisms of action
fumigants,
for example aluminium phosphide, methyl bromide, sulphuryl fluoride
antifeedants,
for example cryolite, flonicamid, pymetrozine
mite growth inhibitors,
for example clofentezine, etoxazole, hexythiazox
amidoflumet, benclothiaz, benzoximate, bifenazate, bromopropylate, buprofezin, chinomethionat, chlordimeform, chlorobenzilate, chloropicrin, clothiazoben, cycloprene, cyflumetofen, dicyclanil, fenoxacrim, fentrifanil, flubenzimine, flufenerim, flutenzin, gossyplure, hydramethylnone, japonilure, metoxadiazone, petroleum, piperonyl butoxide, potassium oleate, pyridalyl, sulfluramid, tetradifon, tetrasul, triarathene, verbutin

A mixture with other known active compounds, such as herbicides, fertilizers, growth regulators, safeners, semiochemicals, or else with agents for improving the plant properties, is also possible.

When used as insecticides, the active compounds according to the invention can furthermore be present in their commercially available formulations and in the use forms, prepared from these formulations, as a mixture with synergists. Synergists are compounds which increase the action of the active compounds, without it being necessary for the synergistic agent added to be active itself.

When used as insecticides, the active compounds according to the invention can furthermore be present in their commercially available formulations and in the use forms, prepared from these formulations, as a mixture with inhibitors which reduce degradation of the active compound after use in the environment of the plant, on the surface of parts of plants or in plant tissues.

The active compound content of the use forms prepared from the commercially available formulations can vary within wide limits. The active compound concentration of the use forms can be from 0.00000001 to 95% by weight of active compound, preferably between 0.00001 and 1% by weight.

The compounds are employed in a customary manner appropriate for the use forms.

All plants and plant parts can be treated in accordance with the invention. Plants are to be understood as meaning in the present context all plants and plant populations such as desired and undesired wild plants or crop plants (including naturally occurring crop plants). Crop plants can be plants which can be obtained by conventional plant breeding and optimization methods or by biotechnological and genetic engineering methods or by combinations of these methods, including the transgenic plants and including the plant cultivars protectable or not protectable by plant breeders' rights. Plant parts are to be understood as meaning all parts and organs of plants above and below the ground, such as shoot, leaf, flower and root, examples which may be mentioned being leaves, needles, stalks, stems, flowers, fruit bodies, fruits, seeds, roots, tubers and rhizomes. The plant parts also include harvested material, and vegetative and generative propagation material, for example cuttings, tubers, rhizomes, offshoots and seeds.

Treatment according to the invention of the plants and plant parts with the active compounds is carried out directly or by allowing the compounds to act on the surroundings, habitat or storage space by the customary treatment methods, for example by immersion, spraying, evaporation, fogging, scattering, painting on, injection and, in the case of propagation material, in particular in the case of seeds, also by applying one or more coats.

The mixtures according to the invention are particularly suitable for treating seed. Here, the combinations according to the invention mentioned above as preferred or particularly preferred may be mentioned as being preferred. Thus, a large part of the damage to crop plants which is caused by pests occurs as early as when the seed is attacked during storage and after the seed is introduced into the soil, during and immediately after germination of the plants. This phase is particularly critical since the roots and shoots of the growing plant are particularly sensitive and even minor damage can lead to the death of the whole plant. Protecting the seed and the germinating plant by the use of suitable compositions is therefore of particularly great interest.

The control of pests by treating the seeds of plants has been known for a long time and is subject-matter of continuous improvements. However, the treatment of seed frequently entails a series of problems which cannot always be solved in a satisfactory manner Thus, it is desirable to develop methods for protecting the seed and the germinating plant which dispense with the additional application of crop protection agents after sowing or after the emergence of the plants. It is furthermore desirable to optimize the amount of active compound employed in such a way as to provide maximum protection for the seed and the germinating plant from attack by pests, but without damaging the plant itself by the active compound employed. In particular, methods for the treatment of seed should also take into consideration the intrinsic insecticidal properties of transgenic plants in order to achieve optimum protection of the seed and the germinating plant with a minimum of crop protection agents being employed.

The present invention therefore in particular also relates to a method for the protection of seed and germinating plants from attack by pests, by treating the seed with a composition according to the invention. The invention likewise relates to the use of the compositions according to the invention for the treatment of seed for protecting the seed and the germinating plant from pests. Furthermore, the invention relates to seed which has been treated with a composition according to the invention so as to afford protection from pests.

One of the advantages of the present invention is that the particular systemic properties of the compositions according to the invention mean that treatment of the seed with these compositions not only protects the seed itself, but also the resulting plants after emergence, from pests. In this manner, the immediate treatment of the crop at the time of sowing or shortly thereafter can be dispensed with.

Furthermore, it must be considered as advantageous that the mixtures according to the invention can also be employed in particular in transgenic seed, the plants arising from this seed being capable of expressing a protein directed against pests. By treating such seed with the compositions according to the invention, certain pests can be controlled merely by the expression of the, for example, insecticidal protein, and additionally be protected by the compositions according to the invention against damage.

The compositions according to the invention are suitable for protecting seed of any plant variety as already mentioned above which is employed in agriculture, in the greenhouse, in forests or in horticulture. In particular, this takes the form of seed of maize, peanut, canola, oilseed rape, poppy, soya beans, cotton, beet (for example sugar beet and fodder beet), rice, sorghum and millet, wheat, barley, oats, rye, sunflower, tobacco, potatoes or vegetables (for example tomatoes, cabbage plants). The compositions according to the invention are likewise suitable for treating the seed of fruit plants and vegetables as already mentioned above. The treatment of the seed of maize, soya beans, cotton, wheat and canola or oilseed rape is of particular importance.

As already mentioned above, the treatment of transgenic seed with a composition according to the invention is also of particular importance. This takes the form of seed of plants which, as a rule, comprise at least one heterologous gene which governs the expression of a polypeptide with in particular insecticidal properties. In this context, the heterologous genes in transgenic seed may be derived from microorganisms such as Bacillus, Rhizobium, Pseudomonas, Serratia, Trichoderma, Clavibacter, Glomus or Gliocladium. The present invention is particularly suitable for the treatment of transgenic seed which comprises at least one heterologous gene orignating from Bacillus sp. and whose gene product shows activity against the European corn borer and/or the corn root worm. It is particularly preferably a heterologous gene derived from Bacillus thuringiensis.

In the context of the present invention, the composition according to the invention is applied to the seed either alone or in a suitable formulation. Preferably, the seed is treated in a state which is stable enough to avoid damage during treatment. In general, the seed may be treated at any point in time between harvest and sowing. The seed usually used has been separated from the plant and freed from cobs, shells, stalks, coats, hairs or the flesh of the fruits.

When treating the seed, care must generally be taken that the amount of the composition according to the invention applied to the seed and/or the amount of further additives is chosen in such a way that the germination of the seed is not adversely affected, or that the resulting plant is not damaged. This must be borne in mind in particular in the case of active compounds which may have phytotoxic effects at certain application rates.

As already mentioned above, it is possible to treat all plants and their parts according to the invention. In a preferred embodiment, wild plant species and plant cultivars, or those obtained by conventional biological breeding methods, such as crossing or protoplast fusion, and parts thereof, are treated. In a further preferred embodiment, transgenic plants and plant cultivars obtained by genetic engineering methods, if appropriate in combination with conventional methods (Genetically Modified Organisms), and parts thereof are treated. The terms “parts”, “parts of plants” and “plant parts” have been explained above.

Particularly preferably, plants of the plant cultivars which are in each case commercially available or in use are treated according to the invention. Plant cultivars are to be understood as meaning plants having novel properties (“traits”) which have been obtained by conventional breeding, by mutagenesis or by recombinant DNA techniques. These can be cultivars, bio- or genotypes.

Depending on the plant species or plant cultivars, their location and growth conditions (soils, climate, vegetation period, diet), the treatment according to the invention may also result in superadditive (“synergistic”) effects. Thus, for example, reduced application rates and/or a widening of the activity spectrum and/or an increase in the activity of the substances and compositions which can be used according to the invention, better plant growth, increased tolerance to high or low temperatures, increased tolerance to drought or to water or soil salt content, increased flowering performance, easier harvesting, accelerated maturation, higher harvest yields, higher quality and/or a higher nutritional value of the harvested products, better storage stability and/or processability of the harvested products are possible, which exceed the effects which were actually to be expected.

The transgenic plants or plant cultivars (obtained by genetic engineering) which are preferably to be treated according to the invention include all plants which, by virtue of the genetic modification, received genetic material which imparted particularly advantageous, useful traits to these plants. Examples of such traits are better plant growth, increased tolerance to high or low temperatures, increased tolerance to drought or to water or soil salt content, increased flowering performance, easier harvesting, accelerated maturation, higher harvest yields, higher quality and/or a higher nutritional value of the harvested products, better storage stability and/or processability of the harvested products. Further and particularly emphasized examples of such traits are a better defence of the plants against animal and microbial pests, such as against insects, mites, phytopathogenic fungi, bacteria and/or viruses, and also increased tolerance of the plants to certain herbicidally active compounds. Examples of transgenic plants which may be mentioned are the important crop plants, such as cereals (wheat, rice), maize, soya beans, potatoes, sugar beet, tomatoes, peas and other vegetable varieties, cotton, tobacco, oilseed rape and also fruit plants (with the fruits apples, pears, citrus fruits and grapes), and particular emphasis is given to maize, soya beans, potatoes, cotton, tobacco and oilseed rape. Traits that are emphasized are in particular increased defence of the plants against insects, arachnids, nematodes and slugs and snails by virtue of toxins formed in the plants, in particular those formed in the plants by the genetic material from Bacillus thuringiensis (for example by the genes CryIA(a), CryIA(b), CryIA(c), CryIIA, CryIIIA, CryIIIB2, Cry9c, Cry2Ab, Cry3Bb and CryIF and also combinations thereof) (referred to hereinbelow as “Bt plants”). Traits that are also particularly emphasized are the increased defence of the plants against fungi, bacteria and viruses by systemic acquired resistance (SAR), systemin, phytoalexins, elicitors and resistance genes and correspondingly expressed proteins and toxins. Traits that are furthermore particularly emphasized are the increased tolerance of the plants to certain herbicidally active compounds, for example imidazolinones, sulphonylureas, glyphosate or phosphinotricin (for example the “PAT” gene). The genes which impart the desired traits in question can also be present in combination with one another in the transgenic plants. Examples of “Bt plants” which may be mentioned are maize varieties, cotton varieties, soya bean varieties and potato varieties which are sold under the trade names YIELD GARD® (for example maize, cotton, soya bean), KnockOut® (for example maize), StarLink® (for example maize), Bollgard® (cotton), Nucotn® (cotton) and NewLeaf® (potato). Examples of herbicide-tolerant plants which may be mentioned are maize varieties, cotton varieties and soya bean varieties which are sold under the trade names Roundup Ready® (tolerance to glyphosate, for example maize, cotton, soya bean), Liberty Link® (tolerance to phosphinotricin, for example oilseed rape), IMI® (tolerance to imidazolinones) and STS® (tolerance to sulphonylureas, for example maize). Herbicide-resistant plants (plants bred in a conventional manner for herbicide tolerance) which may be mentioned include the varieties sold under the name Clearfield® (for example maize) Of course, these statements also apply to plant cultivars having these genetic traits or genetic traits still to be developed, which plant cultivars will be developed and/or marketed in the future.

The plants listed can be treated according to the invention in a particularly advantageous manner with the compounds of the general formula I and/or the active compound mixtures according to the invention. The preferred ranges stated above for the active compounds or mixtures also apply to the treatment of these plants. Particular emphasis is given to the treatment of plants with the compounds or mixtures specifically mentioned in the present text.

The active compounds according to the invention act not only against plant, hygiene and stored product pests, but also in the veterinary medicine sector against animal parasites (ecto- and endoparasites), such as hard ticks, soft ticks, mange mites, leaf mites, flies (biting and licking), parasitic fly larvae, lice, hair lice, feather lice and fleas. These parasites include:

From the order of the Anoplurida, for example, Haematopinus spp., Linognathus spp., Pediculus spp., Phtirus spp., Solenopotes spp.

From the order of the Mallophagida and the suborders Amblycerina and Ischnocerina, for example, Trimenopon spp., Menopon spp., Trinoton spp., Bovicola spp., Werneckiella spp., Lepikentron spp., Damalina spp., Trichodectes spp., Felicola spp.

From the order of the Diptera and the suborders Nematocerina and Brachycerina, for example, Aedes spp., Anopheles spp., Culex spp., Simulium spp., Eusimulium spp., Phlebotomus spp., Lutzomyia spp., Culicoides spp., Chrysops spp., Hybomitra spp., Atylotus spp., Tabanus spp., Haematopota spp., Philipomyia spp., Braula spp., Musca spp., Hydrotaea spp., Stomoxys spp., Haematobia spp., Morellia spp., Fannia spp., Glossina spp., Calliphora spp., Lucilia spp., Chrysomyia spp., Wohlfahrtia spp., Sarcophaga spp., Oestrus spp., Hypoderma spp., Gasterophilus spp., Hippobosca spp., Lipoptena spp., Melophagus spp.

From the order of the Siphonapterida, for example, Pulex spp., Ctenocephalides spp., Xenopsylla spp., Ceratophyllus spp.

From the order of the Heteropterida, for example, Cimex spp., Triatoma spp., Rhodnius spp., Panstrongylus spp.

From the order of the Blattarida, for example, Blatta orientalis, Periplaneta americana, Blattela germanica, Supella spp.

From the subclass of the Acari (Acarina) and the orders of the Meta- and Mesostigmata, for example, Argas spp., Ornithodorus spp., Otobius spp., Ixodes spp., Amblyomma spp., Boophilus spp., Dermacentor spp., Haemophysalis spp., Hyalomma spp., Rhipicephalus spp., Dermanyssus spp., Raillietia spp., Pneumonyssus spp., Sternostoma spp., Varroa spp.

From the order of the Actinedida (Prostigmata) and Acaridida (Astigmata), for example, Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp., Laminosioptes spp.

The active compounds of the formula (I) according to the invention are also suitable for controlling arthropods which infest agricultural productive livestock, such as, for example, cattle, sheep, goats, horses, pigs, donkeys, camels, buffalo, rabbits, chickens, turkeys, ducks, geese and bees, other pets, such as, for example, dogs, cats, caged birds and aquarium fish, and also so-called test animals, such as, for example, hamsters, guinea pigs, rats and mice. By controlling these arthropods, cases of death and reduction in productivity (for meat, milk, wool, hides, eggs, honey etc.) should be diminished, so that more economic and easier animal husbandry is possible by use of the active compounds according to the invention.

The active compounds according to the invention are used in the veterinary sector and in animal husbandry in a known manner by enteral administration in the form of, for example, tablets, capsules, potions, drenches, granules, pastes, boluses, the feed-through process and suppositories, by parenteral administration, such as, for example, by injection (intramuscular, subcutaneous, intravenous, intraperitoneal and the like), implants, by nasal administration, by dermal use in the form, for example, of dipping or bathing, spraying, pouring on and spotting on, washing and powdering, and also with the aid of moulded articles containing the active compound, such as collars, ear marks, tail marks, limb bands, halters, marking devices and the like.

When used for cattle, poultry, pets and the like, the active compounds of the formula (I) can be used as formulations (for example powders, emulsions, free-flowing compositions), which comprise the active compounds in an amount of 1 to 80% by weight, directly or after 100 to 10 000-fold dilution, or they can be used as a chemical bath.

It has furthermore been found that the compounds according to the invention also have a strong insecticidal action against insects which destroy industrial materials.

The following insects may be mentioned as examples and as preferred—but without any limitation:

Beetles, such as Hylotrupes bajulus, Chlorophorus pilosis, Anobium punctatum, Xestobium rufovillosum, Ptilinus pecticornis, Dendrobium pertinex, Ernobius mollis, Priobium carpini, Lyctus brunneus, Lyctus africanus, Lyctus planicollis, Lyctus linearis, Lyctus pubescens, Trogoxylon aequale, Minthes rugicollis, Xyleborus spec. Tryptodendron spec. Apate monachus, Bostrychus capucins, Heterobostrychus brunneus, Sinoxylon spec. Dinoderus minutus;

Hymenopterons, such as Sirex juvencus, Urocerus gigas, Urocerus gigas taignus, Urocerus augur;

Termites, such as Kalotermes flavicollis, Cryptotermes brevis, Heterotermes indicola, Reticulitermes flavipes, Reticulitermes santonensis, Reticulitermes lucifugus, Mastotermes darwiniensis, Zootermopsis nevadensis, Coptotermes formosanus;

Bristletails, such as Lepisma saccharina.

Industrial materials in the present connection are to be understood as meaning non-living materials, such as, preferably, plastics, adhesives, sizes, papers and cardboards, leather, wood and processed wood products and coating compositions.

The ready-to-use compositions may, if appropriate, comprise further insecticides and, if appropriate, one or more fungicides.

With respect to possible additional additives, reference may be made to the insecticides and fungicides mentioned above.

The compounds according to the invention can likewise be employed for protecting objects which come into contact with saltwater or brackish water, such as hulls, screens, nets, buildings, moorings and signalling systems, against fouling.

Furthermore, the compounds according to the invention, alone or in combinations with other active compounds, may be employed as antifouling agents.

In domestic, hygiene and stored-product protection, the active compounds are also suitable for controlling animal pests, in particular insects, arachnids and mites, which are found in enclosed spaces such as, for example, dwellings, factory halls, offices, vehicle cabins and the like. They can be employed alone or in combination with other active compounds and auxiliaries in domestic insecticide products for controlling these pests. They are active against sensitive and resistant species and against all developmental stages.

These pests include:

From the order of the Scorpionidea, for example, Buthus occitanus.

From the order of the Acarina, for example, Argas persicus, Argas reflexus, Bryobia ssp., Dermanyssus gallinae, Glyciphagus domesticus, Ornithodorus moubat, Rhipicephalus sanguineus, Trombicula alfreddugesi, Neutrombicula autumnalis, Dermatophagoides pteronissimus, Dermatophagoides forinae.

From the order of the Araneae, for example, Aviculariidae, Araneidae.

From the order of the Opiliones, for example, Pseudoscorpiones chelifer, Pseudoscorpiones cheiridium, Opiliones phalangium.

From the order of the Isopoda, for example, Oniscus asellus, Porcellio scaber.

From the order of the Diplopoda, for example, Blaniulus guttulatus, Polydesmus spp.

From the order of the Chilopoda, for example, Geophilus spp.

From the order of the Zygentoma, for example, Ctenolepisma spp., Lepisma saccharina, Lepismodes inquilinus.

From the order of the Blattaria, for example, Blatta orientalies, Blattella germanica, Blattella asahinai, Leucophaea maderae, Panchlora spp., Parcoblatta spp., Periplaneta australasiae, Periplaneta americana, Periplaneta brunnea, Periplaneta fuliginosa, Supella longipalpa.

From the order of the Saltatoria, for example, Acheta domesticus.

From the order of the Dermaptera, for example, Forficula auricularia.

From the order of the Isoptera, for example, Kalotermes spp., Reticulitermes spp.

From the order of the Psocoptera, for example, Lepinatus spp., Liposcelis spp.

From the order of the Coleoptera, for example, Anthrenus spp., Attagenus spp., Dermestes spp., Latheticus oryzae, Necrobia spp., Ptinus spp., Rhizopertha dominica, Sitophilus granarius, Sitophilus oryzae, Sitophilus zeamais, Stegobium paniceum.

From the order of the Diptera, for example, Aedes aegypti, Aedes albopictus, Aedes taeniorhynchus, Anopheles spp., Calliphora erythrocephala, Chrysozona pluvialis, Culex quinquefasciatus, Culex pipiens, Culex tarsalis, Drosophila spp., Fannia canicularis, Musca domestica, Phlebotomus spp., Sarcophaga carnaria, Simulium spp., Stomoxys calcitrans, Tipula paludosa.

From the order of the Lepidoptera, for example, Achroia grisella, Galleria mellonella, Plodia interpunctella, Tinea cloacella, Tinea pellionella, Tineola bisselliella.

From the order of the Siphonaptera, for example, Ctenocephalides canis, Ctenocephalides felis, Pulex irritans, Tunga penetrans, Xenopsylla cheopis.

From the order of the Hymenoptera, for example, Camponotus herculeanus, Lasius fuliginosus, Lasius niger, Lasius umbratus, Monomorium pharaonis, Paravespula spp., Tetramorium caespitum.

From the order of the Anoplura, for example, Pediculus humanus capitis, Pediculus humanus corporis, Pemphigus spp., Phylloera vastatrix, Phthirus pubis.

From the order of the Heteroptera, for example, Cimex hemipterus, Cimex lectularius, Rhodinus prolixus, Triatoma infestans.

In the field of household insecticides, they are used alone or in combination with other suitable active compounds, such as phosphoric esters, carbamates, pyrethroids, neonicotinoids, growth regulators or active compounds from other known classes of insecticides.

They are used in aerosols, pressure-free spray products, for example pump and atomizer sprays, automatic fogging systems, foggers, foams, gels, evaporator products with evaporator tablets made of cellulose or polymer, liquid evaporators, gel and membrane evaporators, propeller-driven evaporators, energy-free, or passive, evaporation systems, moth papers, moth bags and moth gels, as granules or dusts, in baits for spreading or in bait stations.

¹H-NMR data

¹H-NMR-data were determined with a Bruker Avance 400 equipped with a flow cell (60 !al volume) or with a Bruker AVIII 400 equipped with 1.7 mm cryo-CPTCI probe head or with a Bruker AVII 600 (600.13 MHz) equipped with a cyroTCI probe head or with a Bruker AVIII 600 (601.6 MHz) equipped with a cryo CPMNP probe head with tetramethylsilane as reference (0.0) and the solvents CD₃CN, CDCl₃, [D₆]-DMSO.

¹H-NMR-data of selected examples are listed in classic format (chemical shift 6, multiplicity, number of hydrogen atoms) or as NMR-peak-lists.

¹H NMR spectra are reported in ppm downfield from tetramethylsilane.

“s” means singlet, “d” means doublet, “dd” means doublet of doublets, “t” means triplet, “q” means quartet, “br s” means broad singlet, “m” means multiplet.

NMR-peak-lists:

If NMR-data of selected examples are provided in form of ¹H-NMR-peak lists, then for every peak first the chemical shift δ in ppm and then, separated by a blank, the intensity of the signal in round brackets is listed. Between the δ-value—signal intensity pairs are semicolons as delimiters.

The peak list of an example is therefore listed as: δ₁ (intensity₁); δ₂(intensity₂); . . . ; δᵢ(intensityᵢ); . . . ; δₙ (intensityₙ).

Intensity of sharp signals correlates with the height of the signals in a printed example of a NMR spectrum in cm and shows the real relations of signal intensities. From broad signals several peaks or the middle of the signal and their relative intensity in comparison to the most intensive signal in the spectrum can be shown.

For calibrating chemical shift for 1H spectra, we use tetramethylsilane and/or the chemical shift of the solvent used, especially in the case of spectra measured in DMSO. Therefore in NMR peak lists, tetramethylsilane peak can occur but not necessarily.

The 1H-NMR peak lists are similar to classical 1H-NMR prints and contain therefore usually all peaks, which are listed at classical NMR-interpretation.

Additionally they can show like classical 1H-NMR prints signals of solvents, stereoisomers of the target compounds, which are also object of the invention, and/or peaks of impurities.

To show compound signals in the delta-range of solvents and/or water the usual peaks of solvents, for example peaks of DMSO in DMSO-D6 and the peak of water are shown in our 1H-NMR peak lists and have usually on average a high intensity.

The peaks of stereoisomers of the target compounds and/or peaks of impurities have usually on average a lower intensity than the peaks of target compounds (for example with a purity >90%).

Such stereoisomers and/or impurities can be typical for the specific preparation process. Therefore their peaks can help to recognize the reproduction of our preparation process via “side-products-fingerprints”.

An expert, who calculates the peaks of the target compounds with known methods (MestreC, ACD-simulation, but also with empirically evaluated expectation values) can isolate the peaks of the target compounds as needed optionally using additional intensity filters. This isolation would be similar to relevant peak picking at classical ¹H-NMR interpretation.

Further details of NMR-data description with peak lists you find in the publication “Citation of NMR Peaklist Data within Patent Applications” of the Research Disclosure Database Number 564025.

The solvent, in which the NMR-spectrum was measured, is specified in squared brackets.

General synthetic methods A-D:

Method A

### Example I-1: 3-[3-[(6-Chloro-5-fluoro-3-pyridinyflmethyl]-2-thiazolidinylidene]-1,1,1-trifluoro-2-propanone

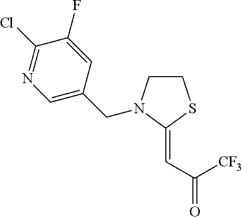 [SMILES: O=C(/C=C1\SCCN1Cc1cnc(Cl)c(F)c1)C(F)(F)F]

200 mg (0,8 mMol) of 2-Chloro-5-chloromethylpyridine and 158,1 mg (0,8 mMol) 1,1,1-trifluoro-3-(2-thiazolidinylidene)-propanone (prepared according to DE 3639877 Al 1988) were dissolved in 5,0 ml N,N-dimethylformamide and 287,4 mg (0,88 mMol) of caesium carbonate were added, and the resulting mixture was stirred at 40° C. for 3 hours. Subsequently, the reaction was cooled at room temperature, filtered and the organic phase was concentrated under reduced pressure. The residue was purified by HPLC (water—acetonitrile gradient neutral) and a fraction including the subject material was collected and concentrated under reduced pressure to obtain 207,6 mg (75,9% yield of theory) of 343-[(6-chloro-5-fluoro-3-pyridinyl)methyl]-2-thiazolidiny lidene]-1,1,1-trifluoro-2-propanone.

LogP-Value (HCOOH)=2,47; 2,48 C₁₂H₉ClF₄N₂OS (340,72 g/mol)

HPLC-MS (ESI Positiv)=341.0 (M⁺)

According to method A the examples I-5, I-7 and I-10 to I-17 can be prepared.

Method B

### Example I-2: 3- [1- [(6-Chloro-5-fluoro-3-pyridinyl)methyl]-2-imidazolidinylidene]-1,1,1-trifluoro-2-propanone

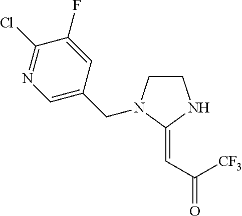 [SMILES: O=C(/C=C1\NCCN1Cc1cnc(Cl)c(F)c1)C(F)(F)F]

Step 1 Preparation of N¹-[(6-chloro-5-fluoro-3-pyridinyl)methyl]-1,2-ethanediamine

10,40 g (46,3 mMol) of 5-(bromomethyl)-2-chloro-3-fluoro-pyridine (prepared according to EP 2633756 A1, 2013) in 90 ml acetonitrile was added at room temperature to 7,57 g (126,0 mMol) ethylenediamine in 160 ml acetonitrile, and the resulting mixture was stirred at 40° C. for 2 hours. Subsequently, the reaction was cooled at room temperature and the reaction mixture was concentrated under reduced pressure. The residue was purified by HPLC (neutral) and a fraction including the subject material was collected and concentrated under reduced pressure to obtain 5,78 g (61,2% yield of theory) of N¹-[(6-chloro-5-fluoro-3-pyridinyl)methyl]-1,2-ethanediamine

Ret. time (HCOOH)=0,61 C₈H₁₁ClFN₃ (203,64 g/mol)

HPLC-MS (ESI Positiv)=204,1 (M⁺)

Step 2

5,78 g (28,3 mMol) of N¹-[(6-chloro-5-fluoro-3-pyridinyl)methyl]-1,2-ethanediamine and 6,02 g (28,3 mMol) 4,4-diethoxy-1,1,1-trifluoro-3-buten-2-one (prepared according to WO 2007/ 067836 A2) were dissolved in 332 ml acetonitrile, and the resulting mixture was stirred at first at room temperature for 18 hours and then further 18 hours under reflux. The reaction mixture was concentrated under reduced pressure and the residue was stirred with 50 ml acetonitrile. The crystals were separated and dried to obtain 5,23 g (55,7% yield of theory; purity 97,9%) of 3-[1-[(6-Chloro-5-fluoro-3-pyridinyl)methyl]-2-imidazolidinylidene]-1,1,1-trifluoro-2-propanone.

LogP-Value (HCOOH)=2,19 C₁₂H₁₀ClF₄N₃O (323,68 g/mol)

HPLC-MS (ESI Positiv)=324.0 (M⁺)

¹H-NMR (600,0 MHz, CD₃CN): σ=9.0755(0.7);8.1695(3.5);8.1672(3.5);7.5924(2.3);7.5891(2.3); 7.5773(2.3);7.5740(2.2);5.4468(0.5);5.1180(8.6);4.4765(16.0);3.7195(3.1);3.7052(5.3);3.7013(2.4);3.68 89(4.4);3.5436(5.1);3.5309(3.0);3.5272(6.6);3.5132(3.4);3.5125(3.3); 2.1361(4.7);1.9647(0.3); 1.9566 (0.8);1.9525(0.9);1.9486(5.0);1.9445(8.7);1.9404(12.6);1.9363(8.5);1.9322(4.2);-0.0001(3.8) ppm.

¹³C-NMR (600 MHz, CD₃CN) σ=43,3 (CH₂—N); 46,3 (CH₂-Pyr); 48,5 (CH₂—N); 71,2 (═CH); 119,7 (CF₃); 135,1 (C-Pyr); 125,2; 145,1 (CH-Pyr); 138,5 (Cl—C-Pyr); 155,6 (F—C-Pyr); 166,0 (═C); 173,0 (C═O) ppm.

According to method B the examples I-6. I-18 and I-19 can be prepared.

Method C

### Example I-3: 3- [3-[(6-Chloro-5-fluoro-3-pyridinyl)methyl]-2-thiazolidinylidene]-1,1,1-trifluoro-2-propanthione

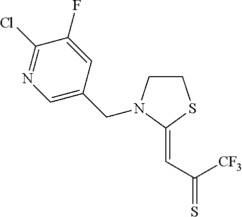 [SMILES: Fc1cc(CN2CCS/C2=C\C(=S)C(F)(F)F)cnc1Cl]

207,6 mg (0,6 mMol) of 3-[14(6-Chloro-5-fluoro-3-pyridinyl)methyl]-2-imidazolidinylidenel-1,1,1-trifluoro-2-propanone (see Example I-1) and 67,7 mg (0,3 mMol) phosphorous pentasulfide and were dissolved in 8 ml dimethoxyethane. The resulting mixture was heated to 65° C. for 1 hour and then led stired at room temperature for 18 hours. Subsequently, the reaction mixture was poured into water and saturated aqueous sodium hydrogencarbonate solution was added. The mixture was extracted twice with ethyl acetate. The combined organic layers were washed with sodium chloride solution, dried over sodium sulfate, filtered and concentrated under vacuum. The cruide material was purified by HPLC (water—acetonitrile gradient neutral) and a fraction including the subject material was collected and concentrated under reduced pressure to obtain 112,4 mg (47,6% yield of theory) of 3-[3-[(6-chloro-5-fluoro-3-pyridiny pmethyl]-2-thiazolidinylidene]-1,1,1-trifluoro-2-propanthione.

LogP-Value (HCOOH)=1,20 C₁₂H₉ClF₄N₂S₂ (356,79 g/mol)

HPLC-MS (ESI Positiv)=357.0 (M⁺)

### Example I-4: 3- [1- [(6-Chloro-5-fluoro-3-pyridinyl)methyl]-2-imidazolidinylidene]-1,1,1-trifluoro-2-propanthione

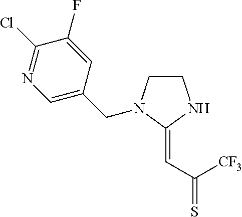 [SMILES: Fc1cc(CN2CCN/C2=C\C(=S)C(F)(F)F)cnc1Cl]

300 mg (0,6 mMol) of 3- [1- [(6-Chloro-5-fluoro-3-pyridinyl)methyl]-2-imidazolidiny lidene]-1,1,1-trifluoro-2-propanone (see Example I-2) and 103 mg (0,46 mMol) phosphorous pentasulfide and were dissolved in 10 ml dimethoxyethane. The resulting mixture was heated to 65° C. for 1 hour and then led stired at room temperature for 18 hours. Subsequently, the reaction mixture was poured into water and saturated aqueous sodium hydrogencarbonate solution was added. The mixture was extracted twice with ethyl acetate. The combined organic layers were washed with sodium chloride solution, dried over sodium sulfate, filtered and concentrated under vacuum. The cruide material was purified by HPLC (water—acetonitrile gradient neutral) and a fraction including the subject material was collected and concentrated under reduced pressure to obtain 134,9 mg (42,8% yield of theory) of 3-[1-[(6-Chloro-5-fluoro-3-pyridiny pmethyl]-2-imidazolidiny lidene]-1,1,1-trifluoro-2-propanthione.

LogP-Value (HCOOH)=2,59/2,61 C₁₂H₁₀ClF₄N₃S (339,74 g/mol)

HPLC-MS (ESI Positiv)=340.1 (M⁺)

According to method C the examples I-8, I-9 and I-20 to I-28 can be prepared.

TABLE 1
Compounds of formula (I)
(I)

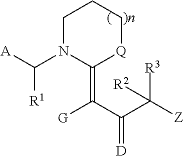

| Example No. | Method | A | R¹ | G | Q/n | 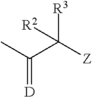 | Yield [in mg] | Purity [in [%] |
|---|---|---|---|---|---|---|---|---|
| I-5 | A | 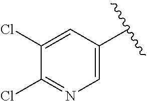 [SMILES: *c1cnc(Cl)c(Cl)c1] | H | H | S/0 | 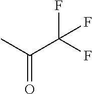 | 408.9 | 85.5 |
| I-6 | B | 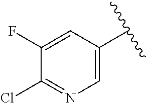 [SMILES: *c1cnc(Cl)c(F)c1] | H | H | NH/1 | 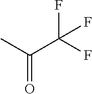 | 366.7 | 99.8 |
| I-7 | A | 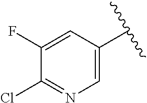 [SMILES: *c1cnc(Cl)c(F)c1] | H | H | NH/0 | 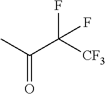 | 460.6 | 100 |
| I-8 | C | 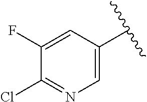 [SMILES: *c1cnc(Cl)c(F)c1] | H | H | S/0 | 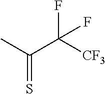 | 109.6 | 100 |
| I-9 | C | 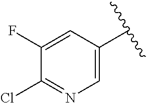 [SMILES: *c1cnc(Cl)c(F)c1] | H | H | NH/0 | 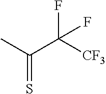 | 67.6 | 89.1 |
| I-10 | A | 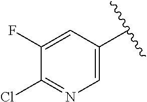 [SMILES: *c1cnc(Cl)c(F)c1] | H | H | S/0 | 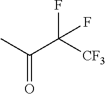 | 91.1 | 100 |
| I-11 | A | 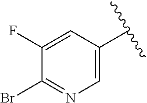 [SMILES: *c1cnc(Br)c(F)c1] | H | H | S/0 | 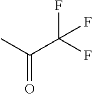 | 62.4 | 100 |
| I-12 | A | 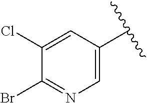 [SMILES: *c1cnc(Br)c(Cl)c1] | H | H | S/0 | 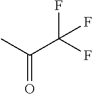 | 83.3 | 100 |
| I-13 | A | 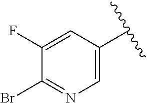 [SMILES: *c1cnc(Br)c(F)c1] | H | H | S/0 | 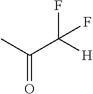 | 37.2 | 100 |
| I-14 | A | 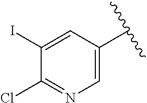 [SMILES: *c1cnc(Cl)c(I)c1] | H | H | S/0 | 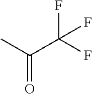 | 33.8 | 100 |
| I-15 | A | 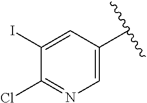 [SMILES: *c1cnc(Cl)c(I)c1] | H | H | S/0 | 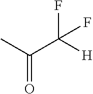 | 25.1 | 90 |
| I-16 | A | 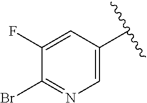 [SMILES: *c1cnc(Br)c(F)c1] | H | H | S/0 | 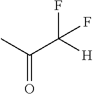 | 44.8 | 100 |
| I-17 | A | 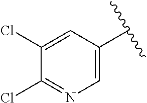 [SMILES: *c1cnc(Cl)c(Cl)c1] | H | H | S/0 | 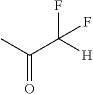 | 125.2 | 100 |
| I-18 | B | 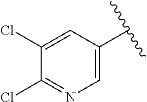 [SMILES: *c1cnc(Cl)c(Cl)c1] | H | H | NH/0 | 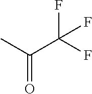 | 53.2 | 97.3 |
| I-19 | B | 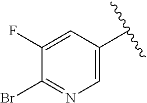 [SMILES: *c1cnc(Br)c(F)c1] | H | H | NH/0 | 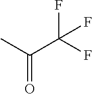 | 60.4 | 100 |
| I-20 | C | 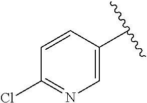 [SMILES: *c1ccc(Cl)nc1] | H | H | S/0 | 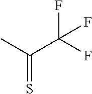 | 40.5 | 100 |
| I-21 | C | 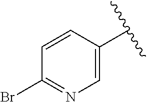 [SMILES: *c1ccc(Br)nc1] | H | H | S/0 | 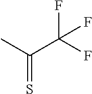 | 29.1 | 96.2 |
| I-22 | C | 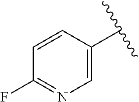 [SMILES: *c1ccc(F)nc1] | H | H | S/0 | 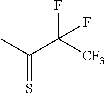 | 79.2 | 99.5 |
| I-23 | C | 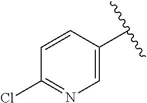 [SMILES: *c1ccc(Cl)nc1] | H | H | S/0 | 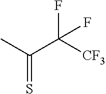 | 47.0 | 100 |
| I-24 | C | 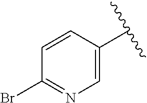 [SMILES: *c1ccc(Br)nc1] | H | H | S/0 | 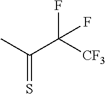 | 8.3 | 89 |
| I-25 | C | 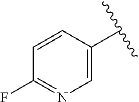 [SMILES: *c1ccc(F)nc1] | H | H | S/0 | 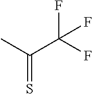 | 73.1 | 100 |
| I-26 | C | 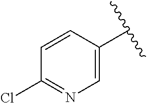 [SMILES: *c1ccc(Cl)nc1] | H | H | NH/0 | 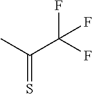 | 38.2 | 95 |
| I-27 | C | 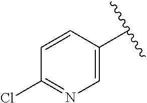 [SMILES: *c1ccc(Cl)nc1] | H | H | NH/0 | 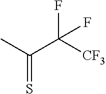 | 30.1 | 72 |
| I-28 | C | 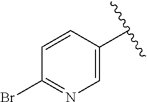 [SMILES: *c1ccc(Br)nc1] | H | H | NH/0 | 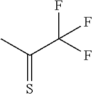 | 29.0 | 100 |

Very specifically disclosed are also those compounds of formula (I) in which:
A refers to 5-fluoro-6-chloropyrid-3-yl or 5,6-dichloropyrid-3-yl
R¹ and G represents hydrogen
Q represents sulfur and NH
n represents 0 or 1
D represents sulfur or oxygen
R² and R³ represents fluorine, and
Z represents fluorine or trifluoromethyl

| Examples | ¹H-NMR-Data |
|---|---|
| I-5 | ¹H-NMR(400.0 MHz, d₆-DMSO): |
| | δ = 8.3696(5.8); 8.3647(6.1); 8.2997(1.0); 8.2949(1.1); 8.0995(7.0); 8.0947(7.0); 5.8690(9.8); 4.8348(16.0); |
| | 4.1244(0.6); 4.1035(1.3); 4.0824(0.7); 3.9222(4.4); 3.9022(8.6); 3.8819(5.0); 3.3178(41.2); 3.2969(1.3); |
| | 3.2795(5.5); 3.2593(9.0); 3.2394(4.5); 2.9674(0.5); 2.9494(1.2); 2.9302(1.0); 2.8313(0.7); 2.8120(1.0); |
| | 2.7949(0.4); 2.6716(0.4); 2.5069(50.2); 2.5027(68.8); 2.4986(53.7); 2.3296(0.4); 0.1461(0.4); 0.0073(2.9); |
| | −0.0002(78.7); −0.1496(0.4) ppm |
| I-13 | ¹H-NMR(600.1 MHz, DMF): |
| | δ = 8.4338(4.9); 8.4303(5.1); 8.1137(5.2); 8.1102(5.3); 8.0248(7.8); 6.1753(2.8); 6.0836(6.1); 5.9919(3.0); |
| | 5.9302(7.6); 4.9012(16.0); 4.0109(4.8); 3.9979(9.1); 3.9847(5.2); 3.4747(11.5); 3.3177(5.4); 3.3046(9.4); |
| | 3.2916(5.0); 2.9523(0.6); 2.9220(3.8); 2.9189(7.8); 2.9157(10.9); 2.9126(8.1); 2.9095(4.1); 2.7841(0.6); |
| | 2.7510(4.1); 2.7478(8.2); 2.7446(11.7); 2.7414(8.5); 2.7382(4.4); −0.0001(6.7) ppm |
| I-16 | ¹H-NMR(600.1 MHz, DMF): |
| | δ = 8.3653(3.3); 8.0249(4.8); 7.9226(1.8); 7.9197(1.8); 7.9078(1.8); 7.9049(1.8); 6.1733(1.8); 6.0816(3.8); |
| | 5.9900(1.9); 5.9180(5.2); 4.9258(11.0); 4.0063(3.3); 3.9933(6.2); 3.9802(3.5); 3.4745(16.0); 3.3185(3.6); |
| | 3.3054(6.4); 3.2924(3.3); 2.9524(0.3); 2.9218(2.3); 2.9188(4.6); 2.9158(6.3); 2.9127(4.6); 2.9097(2.4); |
| | 2.7509(2.4); 2.7478(4.7); 2.7446(6.6); 2.7415(4.8); 2.7384(2.5); −0.0001(3.6) ppm |
| I-17 | ¹H-NMR(600.1 MHz, CD₃CN): |
| | δ = 8.2469(4.0); 8.2435(4.3); 7.8216(4.5); 7.8181(4.7); 5.9225(2.9); 5.8307(5.8); 5.7388(3.0); 5.7296(7.0); |
| | 4.6369(16.0); 3.7995(5.1); 3.7865(9.2); 3.7733(5.5); 3.1959(5.7); 3.1826(9.4); 3.1696(5.2); 2.1513(33.4); |
| | 1.9651(2.2); 1.9570(0.5); 1.9528(0.7); 1.9491(4.4); 1.9450(8.0); 1.9409(11.6); 1.9367(8.1); 1.9326(4.1); |
| | −0.0001(4.1) ppm |

Biological Examples

The following examples illustrate the effectiveness of the compounds of formula (I).

Ctenocephalides felis—in-vitro contact tests adult cat flea

9 mg compound is solved in 1 ml acetone and diluted with acetone to the desired concentration. 250 μl of the test solution is filled in 25ml glass test tubes and homogeneously distributed on the inner walls by rotation and tilting on a shaking device (2 h at 30 rpm). With a compound concentration of 900 ppm, an inner surface of 44,7 cm² and a homogeneous distribution, a dose of 5 μg/cm² is achieved.

After the solvent has evaporated, each test tube is filled with 5-10 adult cat fleas (Ctenocephalides felis), closed with a perforated lid and incubated in a lying position at room temperature and relative humidity. After 48 hours efficacy is determined. The fleas are patted on the ground of the tubes and are incubated on a heating plate at 45-50° C. for at most 5 minutes Immotile or uncoordinated moving fleas, which are not able to escape the heat by climbing upwards, are marked as dead or moribund.

A compound shows a good efficacy against Ctenocephalides felis, if at a compound concentration of 5 μg/cm² an efficacy of at least 80% is monitored. An efficacy of 100% means all fleas are dead or moribund; 0% means no fleas are dead or moribund.

In this test, for example, the following compounds from the preparation examples showed good activity of 100% at an application rate of 5 μg/cm² (=500 g/ha): I-1, I-2, I-4, I-5, I-6, I-20, I-26, I-28

Ctenocephalides felis—oral test (CTECFE)

Solvent: dimethyl sulfoxide

To produce a suitable preparation of active compound, 10 mg of active compound are dissolved in 0.5 ml solvent, and the concentrate is diluted with cattle blood to the desired concentration.

Approximately 20 adult unfed cat fleas (Ctenocephalides felis) are placed in flea chambers. The blood chamber, sealed with parafilm on the bottom, are filled with cattle blood supplied with compound solution and placed on the gauze covered top of the flea chamber, so that the fleas are able to suck the blood. The blood chamber is heated to 37° C. whereas the flea chamber is kept at room temperature.

After 2 days mortality in % is determined. 100% means all the fleas have been killed; 0% means none of the fleas have been killed.

In this test, for example, the following compounds from the preparation examples showed good activity of 100% at an application rate of 100 ppm: I-2

In this test, for example, the following compounds from the preparation examples showed good activity of 95% at an application rate of 100 ppm: I-4

In this test, for example, the following compounds from the preparation examples showed good activity of 90% at an application rate of 100 ppm: I-6, I-26

In this test, for example, the following compounds from the preparation examples showed good activity of 85% at an application rate of 100 ppm: I-19

In this test, for example, the following compounds from the preparation examples showed good activity of 80% at an application rate of 100 ppm: I-28.

Lucilia cuprina—test (LUCICU)

Solvent: dimethyl sulfoxide

10 mg active compound are dissolved in 0,5 ml Dimethylsulfoxid. Serial dilutions are made to obtain the desired rates.

Approximately 20 1^{st} instar larvae of the Australian sheep blowfly (Lucilia cuprina) are transferred into a test tube containing minced horse meat and compound solution of the desired concentration.

After 2 days mortality in % is determined. 100% means all the larvae have been killed; 0% means none of the larvae have been killed.

In this test, for example, the following compounds from the preparation examples showed good activity of 100% at an application rate of 100 ppm: I-1, I-2, I-3, I-4, I-5, I-6, I-19, I-20, I-21, I-26, I-28.

Musca domestica—test (MUSCDO)

Solvent: dimethyl sulfoxide

To produce a suitable preparation of active compound, 10 mg of active compound are dissolved in 0.5 ml solvent, and the concentrate is diluted with water to the desired concentration.

10 adult house flies (Musca domestica) are transferred into a container, containing a sponge soaked with a mixture of sugar solution and compound solution of the desired concentration.

After 2 days mortality in % is determined. 100% means all the flies have been killed; 0% means none of the flies have been killed.

In this test, for example, the following compounds from the preparation examples showed good activity of 100% at an application rate of 20 ppm: I-2, I-19

In this test, for example, the following compounds from the preparation examples showed good activity of 90% at an application rate of 20 ppm: I-1

In this test, for example, the following compounds from the preparation examples showed good activity of 85% at an application rate of 20 ppm: I-26

In this test, for example, the following compounds from the preparation examples showed good activity of 80% at an application rate of 20 ppm: I-6, I-28.

Diabrotica balteata—spray test

| | |
|---|---|
| Solvent: | 78.0 parts by weight of acetone |
| | 1.5 parts by weight of dimethylformamide |
| Emulsifier: | alkylarylpolyglycol ether |

To produce a suitable preparation of active compound, 1 part by weight of active compound is mixed with the stated amount of solvent, and the concentrate is diluted with water, containing an emulsifier concentration of 1000 ppm, to the desired concentration. Further test concentrations are prepared by dilution with emulsifier containing water.

Soaked wheat seeds (Trificum aestivum) are placed in a multiple well plate filled with agar and some water and are incubated for 1 day to germinate (5 seeds per well). The germinated wheat seeds are sprayed with a test solution containing the desired concentration of the active ingredient. Afterwards each unit is infected with 10-20 larvae of the banded cucumber beetle (Diabrofica balteata).

After 7 days efficacy in % is determined. 100% means all the seedlings have grown up like in the untreated, uninfected control; 0% means none of the seedlings have grown.

In this test, for example, the following compounds from the preparation examples showed good activity of 100% at an application rate of 160 gg/well: I-1, I-2, I-3, I-4, I-6, I-7, I-9, I-10, I-13, I-15, I-20, I-21, I-22, I-23, I-24, I-25, I-26, I-27, I-28

In this test, for example, the following compounds from the preparation examples showed good activity of 80% at an application rate of 160 gg/well: I-8

Myzus persicae—spray test

| | |
|---|---|
| Solvent: | 78.0 parts by weight acetone |
| | 1.5 parts by weight dimethylformamide |
| Emulsifier: | alkylarylpolyglycol ether |

To produce a suitable preparation of active compound, 1 part by weight of active compound is mixed with the stated amount of solvents and is diluted with water, containing an emulsifier concentration of 1000 ppm, to the desired concentration. Further test concentrations are prepared by dilution with emulsifier containing water.

Chinese cabbage (Brassica pekinensis) leaf disks infected with all instars of the green peach aphid (Myzus persicae), are sprayed with a preparation of the active ingredient of the desired concentration.

After 5-6 days mortality in % is determined. 100% means all aphids have been killed and 0% means none of the aphids have been killed.

In this test, for example, the following compounds from the preparation examples showed good activity of 100% at an application rate of 100 g/ha: I-1, I-2, I-11, I-12, I-19

In this test, for example, the following compounds from the preparation examples showed good activity of 90% at an application rate of 100 g/ha: I-5, I-6, I-16, I-17, I-18, I-20, I-26.

Myzus persicae—oral test

| | |
|---|---|
| Solvent: | 100 parts by weight acetone |

To produce a suitable preparation of active compound, 1 part by weight of active compound is mixed with the stated amount of solvent, and the concentrate is diluted with water to the desired concentration.

50 μl compound solution is filled in microtiter plates and 150 μl IPL41 insect medium (33% +15% sugar) is added to obtain a total volume of 200 μ per well. Afterwards the plates are sealed with parafilm through which a mixed population of the green peach aphid (Myzus persicae) can suck on the compound preparation.

After 5 days mortality in % is determined. 100% means all aphids have been killed and 0% means none of the aphids have been killed.

In this test, for example, the following compounds from the preparation examples showed good activity of 100% at an application rate of 20 ppm: I-3, I-5, I-7, I-10, I-20, I-21, I-25, I-26, I-28

In this test, for example, the following compounds from the preparation examples showed good activity of 100% at an application rate of 4 ppm: I-3, I-4, I-5, I-10, I-20, I-21, I-26, I-28.

Phaedon cochleariae—spray test

| | |
|---|---|
| Solvent: | 78.0 parts by weight of acetone |
| | 1.5 parts by weight of dimethylformamide |
| Emulsifier: | alkylarylpolyglycol ether |

To produce a suitable preparation of active compound, 1 part by weight of active compound is mixed with the stated amount of solvents and is diluted with water, containing an emulsifier concentration of 1000 ppm, to the desired concentration. Further test concentrations are prepared by dilution with emulsifier containing water.

Chinese cabbage (Brassica pekinensis) leaf disks are sprayed with a preparation of the active ingredient of the desired concentration. Once dry, the leaf disks are infested with mustard beetle larvae (Phaedon cochleariae).

After 7 days mortality in % is determined. 100% means all beetle larvae have been killed and 0% means none of the beetle larvae have been killed.

In this test, for example, the following compounds from the preparation examples showed good activity of 100% at an application rate of 500 g/ha: I-20, I-26, I-28

In this test, for example, the following compounds from the preparation examples showed good activity of 83% at an application rate of 500 g/ha: I-4, I-5, I-7, I-10

Tetranvchus urticae—spray test OP-resistant

| | |
|---|---|
| Solvent: | 78.0 parts by weight acetone |
| | 1.5 parts by weight dimethylformamide |
| Emulsifier: | alkylarylpolyglycol ether |

To produce a suitable preparation of active compound, 1 part by weight of active compound is mixed with the stated amount of solvents and is diluted with water, containing an emulsifier concentration of 1000 ppm, to the desired concentration. Further test concentrations are prepared by dilution with emulsifier containing water.

French bean (Phaseolus vulgaris) leaf disks infected with all instars of the two spotted spidermite (Tetranychus urticae), are sprayed with a preparation of the active ingredient of the desired concentration.

After 6 days mortality in % is determined. 100% means all spider mites have been killed and 0% means none of the spider mites have been killed.

In this test, for example, the following compounds from the preparation examples showed good activity of 90% at an application rate of 100g/ha: I-18

Nilaparvata Wens—spray test

| | |
|---|---|
| Solvent: | 78.0 parts by weight of acetone |
| | 1.5 parts by weight of dimethylformamide |
| Emulsifier: | alkylarylpolyglycol ether |

To produce a suitable preparation of active compound, 1 part by weight of active compound is mixed with the stated amount of solvents and is diluted with water, containing an emulsifier concentration of 1000 ppm, to the desired concentration. Further test concentrations are prepared by dilution with emulsifier containing water.

Rice plants (Oryza sativa) are sprayed with a preparation of the active ingredient of the desired concentration and the plants are infested with the brown planthopper (Nilaparvata lugens).

After 4 days mortality in % is determined. 100% means all planthoppers have been killed and 0% means none of the planthoppers have been killed.

In this test, for example, the following compounds from the preparation examples showed good activity of 100% at an application rate of 500g/ha: I-3, I-4, I-7, I-10, I-20, I-25, I-26, I-28

In this test, for example, the following compounds from the preparation examples showed good activity of 90% at an application rate of 500g/ha: I-22

Nezara viridula—spray test

| | |
|---|---|
| Solvent: | 78.0 parts by weight of acetone |
| | 1.5 parts by weight of dimethylformamide |
| Emulsifier: | alkylarylpolyglycol ether |

To produce a suitable preparation of active compound, 1 part by weight of active compound is mixed with the stated amount of solvent, and the concentrate is diluted with water, containing an emulsifier concentration of 1000 ppm, to the desired concentration. Further test concentrations are prepared by dilution with emulsifier containing water.

Barley plants (Hordeum vulgare) infested with larvae of the southern green stink bug (Nezara viridula) are sprayed with a test solution containing the desired concentration of the active ingredient.

After 4 days mortality in % is determined. 100% means all the stink bugs have been killed; 0% means none of the stink bugs have been killed.

In this test, for example, the following compounds from the preparation examples showed good activity of 100% at an application rate of 500g/ha: I-10

In this test, for example, the following compounds from the preparation examples showed good activity of 90% at an application rate of 500g/ha: I-26, I-28

Nezara viridula—spray test

| | |
|---|---|
| Solvent: | 52.5 parts by weight of acetone |
| | 7 parts by weight of dimethylformamide |
| Emulsifier: | alkylaryl polyglycolether |

To produce a suitable preparation of active compound, 1 part by weight of active compound is mixed with the stated amount of solvent, and the concentrate is diluted with water, containing an emulsifier concentration of 1000 ppm, to the desired concentration Ammonium salt and/or penetration enhancer in a dosage of 1000 ppm are added to the desired concentration if necessary.

Barley plants (Hordeum vulgare) infested with larvae of the southern green stink bug (Nezara viridula) are sprayed with a test solution containing the desired concentration of the active ingredient.

After 4 days mortality in % is determined. 100% means all the stink bugs have been killed; 0% means none of the stink bugs have been killed.

In this test, for example, the following compounds from the preparation examples showed good activity of 100% at an application rate of 500 g/ha: I-1, I-6, I-19

In this test, for example, the following compounds from the preparation examples showed good activity of 90% at an application rate of 500 g/ha: I-2, I-20.

Nilaparvata Wens—spray test

| | |
|---|---|
| Solvent: | 52.5 parts by weight of acetone |
| | 7 parts by weight of dimethylformamide |
| Emulsifier: | alkylaryl polyglycolether |

To produce a suitable preparation of active compound, 1 part by weight of active compound is mixed with the stated amount of solvents and is diluted with water, containing an emulsifier concentration of 1000 ppm, to the desired concentration. Further test concentrations are prepared by dilution with emulsifier containing water Ammonium salt and/or penetration enhancer in a dosage of 1000 ppm are added to the desired concentration if necessary.

Rice plants (Oryza sativa) are treated by being sprayed with the desired concentration of the active compound and are infested with larvae of the brown planthopper (Nilaparvata lugens).

After 4 days mortality in % is determined. 100% means all planthoppers have been killed and 0% means none of the planthoppers have been killed.

In this test, for example, the following compounds from the preparation examples showed good activity of 100% at an application rate of 500g/ha: I-1, I-2, I-6, I-19, I-20.

1. A compound of formula (I), 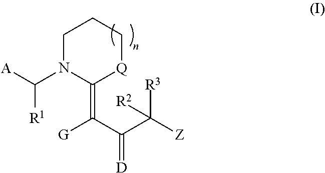
in which
A represents a pyridinyl, pyrimidinyl, pyrazolyl, thiophenyl, oxazolyl, isoxazolyl, 1,2,4-oxadiazolyl, thiazolyl, isothiazolyl, 1,2,4-triazolyl or 1,2,5-thiadiazolyl radical which is substituted by at least one fluorine, chlorine, bromine, cyano, nitro, C₁-C₄-alkyl (which is optionally substituted by fluorine and/or chlorine), C₁-C₃-alkylthio (which is optionally substituted by fluorine and/or chlorine), or C₁-C₃-alkylsulphonyl (which is optionally substituted by fluorine and/or chlorine), and
n represents 0 or 1, and
D represents sulphur, and
G represents hydrogen, and
Q represents sulphur, oxygen or NH, and
R¹ represents hydrogen or alkyl, and
R² represents halogen, and
R³ represents halogen, and
Z represents hydrogen, halogen or halogenalkyl,
or in which
A represents a radical 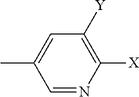
and in which
X represents halogen, alkyl or haloalkyl, and
Y represents halogen, alkyl, haloalkyl, haloalkoxy, azido or cyano, and
n represents 0 or 1, and
D represents oxygen or sulphur, and
G represents hydrogen or halogen, and
Q represents sulphur, oxygen or NH, and
R¹ represents hydrogen or alkyl, and
R² represents halogen, and
R³ represents halogen, and
Z represents hydrogen, halogen or halogenalkyl.

2. A compound according to claim 1, in which
A represents pyrimidin-5-yl which is optionally substituted in the 2-position by halogen or C₁-C₄-alkyl, represents 1H-pyrazol-4-yl which is optionally substituted in the 1-position by C₁-C₄-alkyl and in the 3-position by halogen, represents 1H-pyrazol-5-yl which is optionally substituted in the 2-position by halogen or C₁-C₄-alkyl, represents isoxazol-5-yl which is optionally substituted in the 3-position by halogen or C₁-C₄-alkyl, represents 1,2,4-oxadiazol-5-yl which is optionally substituted in the 3-position by halogen or C₁-C₄-alkyl, represents 1-methyl-1,2,4-triazol-3-yl or represents 1,2,5-thiadiazol-3-yl, or
n represents 0 or 1.
D represents sulfur,
G represents hydrogen,
Q represents sulphur or NH,
R¹ represents hydrogen,
R² represents halogen,
R³ represents halogen,
Z represents halogen or halogenalkyl.

3. A compound according to claim 1, in which
A represents one of the radicals 5,6-difluoropyrid-3-yl, 5-chloro-6-fluoropyrid-3-yl, 5-bromo-6-fluoropyrid-3-yl, 5-iodo-6-fluoropyrid-3-yl, 5-fluoro-6-chloropyrid-3-yl, 5, 6-dichloropyrid-3-yl, 5-bromo-6-chloropyrid-3-yl, 5-iodo-6-chloropyrid-3-yl, 5-fluoro-6-bromopyrid-3-yl, 5-chloro-6-bromopyrid-3-yl, 5, 6-dibromopyrid-3-yl, 5-iodo-6-bromopyrid-3-yl, 5-fluoro-6-iodopyrid-3-yl, 5-chloro-6-iodopyrid-3-yl, 5-bromo-6-iodopyrid-3-yl, 5, 6-diiodopyrid-3-yl, 5-methyl-6-fluoropyrid-3-yl, 5-methyl-6-chloropyrid-3-yl, 5-methyl-6-bromopyrid-3-yl, 5-methyl-6-iodopyrid-3-yl, 5-difluoromethyl-6-fluoropyrid-3-yl, 5-difluoromethyl-6-chloropyrid-3-yl, 5-difluoromethyl-6-bromopyrid-3-yl, 5-difluoromethyl-6-iodopyrid-3-yl .
n represents 0 or 1.
D represents oxygen,
G represents hydrogen,
Q represents sulphur or NH,
R¹ represents hydrogen,
R² represents halogen,
R³ represents halogen,
Z represents halogen or halogenalkyl.

4. A compound of formula (I), 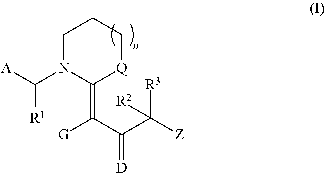
in which
A represents a pyridinyl, pyrimidinyl, pyrazolyl, thiophenyl, oxazolyl, isoxazolyl, 1,2,4-oxadiazolyl, thiazolyl, isothiazolyl, 1,2,4-triazolyl or 1,2,5-thiadiazolyl radical which is substituted by at least one fluorine, chlorine, bromine, cyano, nitro, C₁-C₄-alkyl (which is optionally substituted by fluorine and/or chlorine), C₁-C₃-alkylthio (which is optionally substituted by fluorine and/or chlorine), or C₁-C₃-alkylsulphonyl (which is optionally substituted by fluorine and/or chlorine), and
n represents 0 or 1, and
D represents sulphur, and
G represents hydrogen, and
Q represents sulphur, oxygen or NH, and
R¹ represents hydrogen or alkyl, and
R² represents halogen, and
R³ represents halogen, and
Z represents hydrogen, halogen or halogenalkyl

5. A compound of formulae (I-A-a), (I-A-b), (I-B-a), (I-B-b), 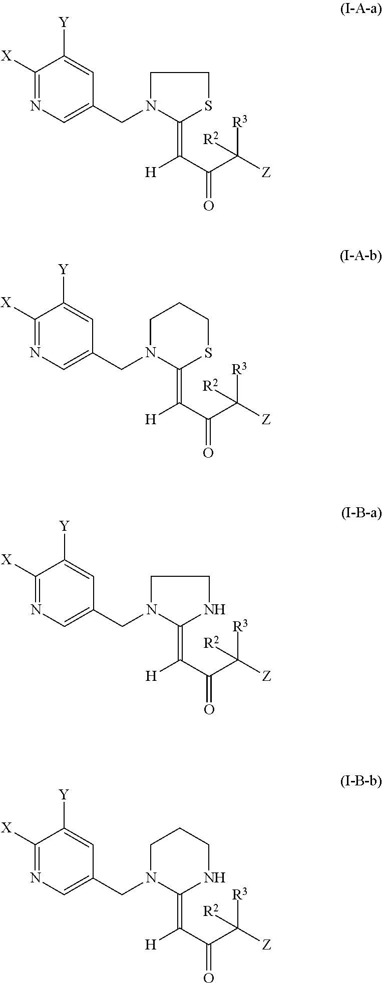
in which
A represents a pyridinyl, pyrimidinyl, pyrazolyl, thiophenyl, oxazolyl, isoxazolyl, 1,2,4-oxadiazolyl, thiazolyl, isothiazolyl, 1,2,4-triazolyl or 1,2,5-thiadiazolyl radical which is substituted by at least one fluorine, chlorine, bromine, cyano, nitro, C₁-C₄-alkyl (which is optionally substituted by fluorine and/or chlorine), C₁-C₃-alkylthio (which is optionally substituted by fluorine and/or chlorine), or C₁-C₃-alkylsulphonyl (which is optionally substituted by fluorine and/or chlorine), and
n represents 0 or 1, and
D represents sulphur, and
G represents hydrogen, and
Q represents sulphur, oxygen or NH, and
R¹ represents hydrogen or alkyl, and
R² represents halogen, and
R³ represents halogen, and
Z represents hydrogen, halogen or halogenalkyl,
or in which
A represents a radical 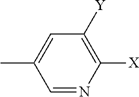
and in which
X represents halogen, alkyl or haloalkyl, and
Y represents halogen, alkyl, haloalkyl, haloalkoxy, azido or cyano, and
n represents 0 or 1, and
D represents oxygen or sulphur, and
G represents hydrogen or halogen, and
Q represents sulphur, oxygen or NH, and
R¹ represents hydrogen or alkyl, and
R² represents halogen, and
R³ represents halogen, and
Z represents hydrogen, halogen or halogenalkyl.

6. compound of formulae (I-D-a), (I-D-b), (I-D-c), (I-D-d), 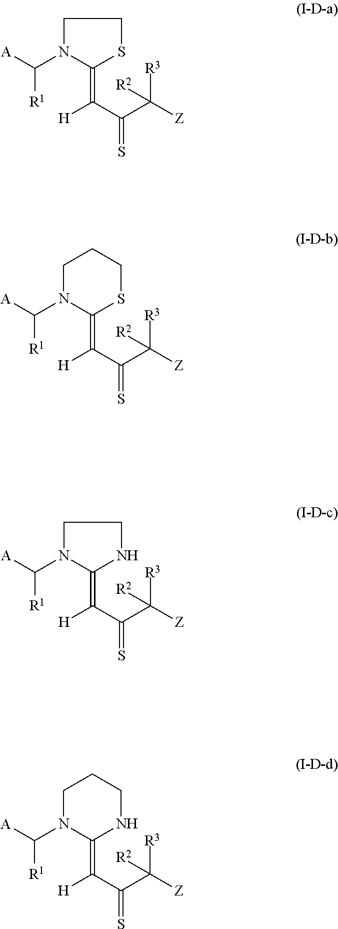
in which
A represents a pyridinyl, pyrimidinyl, pyrazolyl, thiophenyl, oxazolyl, isoxazolyl, 1,2,4-oxadiazolyl, thiazolyl, isothiazolyl, 1,2,4-triazolyl or 1,2,5-thiadiazolyl radical which is substituted by at least one fluorine, chlorine, bromine, cyano, nitro, C₁-C₄-alkyl (which is optionally substituted by fluorine and/or chlorine), C₁-C₃-alkylthio (which is optionally substituted by fluorine and/or chlorine), or C₁-C₃-alkylsulphonyl (which is optionally substituted by fluorine and/or chlorine), and
n represents 0 or 1, and
D represents sulphur, and
G represents hydrogen, and
Q represents sulphur, oxygen or NH, and
R¹ represents hydrogen or alkyl, and
R² represents halogen, and
R³ represents halogen, and
Z represents hydrogen, halogen or halogenalkyl,
or in which
A represents a radical 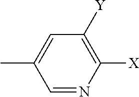
and in which
X represents halogen, alkyl or haloalkyl, and
Y represents halogen, alkyl, haloalkyl, haloalkoxy, azido or cyano, and
n represents 0 or 1, and
D represents oxygen or sulphur, and
G represents hydrogen or halogen, and
Q represents sulphur, oxygen or NH, and
R¹ represents hydrogen or alkyl, and
R² represents halogen, and
R³ represents halogen, and
Z represents hydrogen, halogen or halogenalkyl.
claim 1.

7. A compound of formula (I) of claim 1, in which
A refers to 5-fluoro-6-chloropyrid-3-yl or 5,6-dichloropyrid-3-yl,
R¹ and G represent hydrogen,
Q represents sulfur and NH,
n represents 0 or 1,
D represents sulfur or oxygen,
R² and R³ represents fluorine, and
Z represents fluorine or trifluoromethyl.

8. Composition, comprising at least one compound of formula (I) according to claim 1 and one or more customary extenders and/or surfactants.

9. Method for controlling one or more pests, comprising allowing a compound of formula (I) according to claim 1 or a composition thereof to act on the pests and/or a habitat thereof.

10. A product comprising a compound of formula (I) according to claim 1 or a composition thereof for controlling pests.

11. A product comprising a compound of formula (I) according to claim 1 or a composition thereof for protecting propagation material of one or more plants.
